(19) United States
(12) Patent Application Publication
(10) Pub. No.: US 2011/0144503 A1
(43) Pub. Date: Jun. 16, 2011

(54) Optical methods for monitoring of birefringent tissues

(75) Inventors: Martin P. Debreczeny, Danville, CA (US); Diana Villegas, Alameda, CA (US)

(73) Assignee: Alpha Orthopaedics, Inc., Hayward, CA (US)

(21) Appl. No.: 12/806,811

(22) Filed: Aug. 19, 2010

Related U.S. Application Data
(63) Continuation-in-part of application No. 12/380,014, filed on Feb. 20, 2009.
(60) Provisional application No. 61/066,593, filed on Feb. 20, 2008.
(60) Provisional application No. 61/274,704, filed on Aug. 19, 2009.

(51) Int. Cl. A61B 6/00

(52) U.S. Cl. 600/476

(57) ABSTRACT

The present invention comprises methods and systems/devices for non-invasively measuring birefringent tissues (e.g. collagen) and changes during treatment of tissue, e.g., denaturation by the application of RF energy, through linear dichroism, circular dichroism, or birefringence. The invention optionally uses polarization sensitive optical measurements to discriminate between denaturation of unidirectionally oriented strands of collagen, such as a ligament or tendon, and denaturation of planar collagen surfaces, such as the dermal layer of the skin or collagen in joint capsules.

### CROSS REFERENCE TO RELATED APPLICATION

This application is a Continuation in Part of U.S. Ser. No. 12/380,014, filed Feb. 20, 2009, which claims priority to and benefit of U.S. Provisional Patent Application Ser. No. 61/066,593, filed on Feb. 20, 2008; and claims priority to and benefit of U.S. Provisional Patent Application Ser. No. 61/274,704 filed on Aug. 19, 2009, the disclosures of all of which are incorporated herein in their entireties for all purposes.

### FIELD OF THE INVENTION

The current invention relates to the field of medical technology. More specifically, the present invention provides methods and devices/systems for real time monitoring of tissue treatment, as well as for differentiating between treatment effects on multiple tissue types and/or layers (e.g., unidirectionally oriented collagen and planar collagen).

### BACKGROUND OF THE INVENTION

A major challenge in modern medicine concerns the controlled treatment of biological tissues through application of temperature change. Numerous medical conditions exist which can optionally be treated through use of such thermotherapy. Such treatments hold special promise for modification of collagen fibers both in planar arrangements (e.g., in dermal layers) and in unidirectional strands (e.g., in tendons and ligaments). A wide range of treatment procedures has been, and continues to be, developed to utilize thermotherapy.

While thermotherapy and related treatment regimes have the potential for wide ranging application, they also, however, have the disadvantage in that it is difficult to track their progress. In other words, the extent of desired tissue modification (e.g., collagen denaturation of a specific area) and the extent of undesired tissue modification (e.g., damage to adjacent tissues, etc.) are difficult or impossible to track even in applications where there is direct visualization of the tissues being treated. Furthermore, lack of real time monitoring of such modifications is even more problematic.

Thus, there is a continuing need for better and more controllable methods and systems/devices to allow monitoring, especially real time monitoring, of tissue treatments such as thermotherapy. The current invention provides these and other benefits which will be apparent upon examination.

### SUMMARY OF THE INVENTION

In various embodiments herein, the invention comprises methods of monitoring a change in one or more structures (e.g., collagen) in a tissue (e.g., skin, a capsule, a vascular wall, a vaginal or urethral wall, etc.) through exposing the tissue and thus, the structure(s) to light and measuring the light reflected from one or more structures in the tissue; exposing the structures to treatment which could putatively alter them (e.g., by denaturing them); exposing the treated structures to light again and measuring the light reflected from the treated structures; and comparing the light reflected from the structures before treatment and the light reflected from the structures during or after treatment. In particular embodiments, the tissue can comprise a first and a second structure (e.g., an overlaying structure such as dermal collagen, mucosal collagen, synovial collagen, etc. and an underlying or deeper structure such as a tendon, a ligament, a fascia, or an aponeurosis, etc.). In the various embodiments, the different structures can be monitored simultaneously or sequentially or only one of the structures can be monitored.

In some embodiments herein, the invention comprises methods to determine the minimum Degree of Linear Polarization (DoLP) of a biological structure. In such methods, the structure is exposed to a first light at a first polarization angle and the birefringence of the light reflected back from the structure is measured. The reflected light is measured at four different angles of a detection polarizer (L₄₅, L₊₄₅, I₀, and I₊₉₀). From the reflected light, Q, U, and I are calculated by the equations: Q=I₀−I₉₀, U=I₊₄₅−I₋₄₅, and I=I₀+I₉₀ and the DoLP from the reflected birefringence is calculated as:

$$DoLP = \frac{\sqrt{{Q}^{2} + {U}^{2}}}{I} .$$

The structure being monitored is then exposed to a second light at a slightly different polarization angle than the first exposure light (which second exposure light can be, e.g., 1 degree greater, 2 degrees greater, 3 degrees greater, etc. than the first exposure light) and another DoLP is calculated. In such embodiments, such steps are repeated (i.e., exposure of the structure to light—calculation of DoLP at that exposure—exposure of the structure to a light of slightly different polarization angle, and so on) over a range of 80 or more degrees. The minimum DoLP of the biological structure thus corresponds to the lowest measured DoLP value from this process. The minimum DoLP can substantially correspond to an orientation angle of 45 degrees relative to the birefringence axis orientation of the biological structure being monitored.

In some embodiments, the invention comprises monitoring a change in birefringence status of a biological structure, which change in birefringence status corresponds to a structural or physical change in the biological structure. In such embodiments, a first minimum DoLP is determined as outlined above. Next the structure being monitored (e.g. a tendon of collagen within a tissue) is subjected to treatment (e.g., thermotherapy) and second minimum DoLP is determined as outlined above. The first minimum DoLP is then compared with the second minimum DoLP. Again, a difference between the measured minimum DoLPs, thus, indicates a change in birefringent status of the structure, e.g., due to a change in the structure of the biological structure being monitored (e.g., denaturation) due to the treatment received.

In other embodiments, the invention includes methods of determining the maximum Linear Polarization Angle (θ) of a biological structure (e.g., the sample induced rotation of the linear polarization angle). In such methods, the structure is exposed to a first light at a first polarization angle and a reflected birefringence that is reflected from the structure is measured at L₄₅, L₊₄₅, I₀, and L₊₉₀, wherein I_{φ}, is the reflected birefringence measured by a detection polarizer at polarization angle φ. From the four different measurements of the reflected birefringence, Q, U, and I are computed (Q=I₀−I₉₀, U=I₊₄₅−I₋₄₅, and I=I₀+I₉₀) and θ is calculated from the equation 2θ=tan⁻¹(U/Q). In such embodiments, the structure is exposed to a second light at a second polarization angle (which is greater than the first angle of the first light) and another θ is calculated. Such steps are repeated, each with an incrementally different (e.g., greater) polarization angle until a range of at least 80 degrees is covered. The maximum θ of the biological structure thus corresponds to the greatest value of θ measured. In such embodiments, the maximum θ substantially corresponds to an orientation angle of 45 degrees relative to the birefringence axis orientation of the biological structure.

In some embodiments, the invention comprises monitoring a change in birefringence status of a biological structure, which change in birefringence status corresponds to a structural or physical change in the biological structure. In such embodiments, a first maximum θ is determined as outlined above. The structure being monitored (e.g., a tendon within a tissue) is then subjected to treatment (e.g., thermotherapy) and a second maximum θ is found as described above. The first maximum θ is then compared with the second maximum θ. The difference, if any, between the measured maximum θs, thus, indicates a change in birefringent status of the structure, e.g., due to a physical or structural change from the treatment in the structure being monitored.

In some embodiments herein, the invention includes methods of determining the minimum Degree of Vertical Polarization (DoVP) and/or minimum Degree of Horizontal Polarization (DoHP) of a biological structure. In such methods the structure is exposed to a first light at a first polarization angle; a birefringence that is reflected from the structure is then measured (at angle at L₄₅, L₊₄₅, I₀, and L₊₉₀, wherein I_{φ} is the reflected birefringence measured by a detection polarizer at polarization angle φ); Q, U, and I are computed from the measured reflected birefringence by determining Q=I₀−I₉₀, U=I₊₄₅−I₋₄₅, and I=I₀+I₉₀; a DoVP and/or a DoHP is determined from the measured reflected birefringence, wherein the

$$DoVP = \frac{Q}{I}$$

and the

$$DoHP = \frac{U}{I} ;$$

the structure is exposed to a second light at a second polarization angle (which is greater than the first angle); and another DoVP and/or DoHP is calculated. Such exposure and calculation steps are then repeated over a range of 80 or more degrees. In such methods, the minimum DoVP and/or minimum DoHP of the biological structure corresponds to the lowest value of DoVP and/or DoHP measured and the minimum DoVP and/or minimum DoHP substantially corresponds to an orientation angle of 45 degrees relative to the birefringence axis orientation of the biological structure.

In other embodiments, the invention includes monitoring a change in birefringence status of a biological structure (which change corresponds with a structural or physical change in the biological structure due to a treatment). In such embodiments, a first minimum DoVP and/or DoHP is determined as described above. The structure being monitored (e.g., a tendon or a layer or planar collagen) is then subjected to treatment (e.g., thermotherapy) and a second minimum DoVP and/or DoHP is determined (again as described above). The first minimum DoVP and/or DoHP and the second minimum DoVP and/or DoHP are then compared. Any difference between the two measurements can thus indicate a change in the birefringent status of the structure being monitored due to the treatment received.

In other embodiments, the invention includes determination of the angular dependence of a birefringence of a biological structure relative to an input polarization. Such embodiments include exposing the biological structure to a first light at a first polarization angle; measuring a reflected birefringence that is reflected from the biological structure at the first polarization angle at L₄₅, L₊₄₅, I₀, and L₊₉₀, wherein I_{φ} is the reflected birefringence measured by a detection polarizer at polarization angle φ; computing Q, U, and I from the measured reflected birefringence, wherein Q=I₀−I₉₀, U=I₊₄₅−I₋₄₅, and I=I₀+I₉₀; computing a DoLP, θ, DoVP, or DoHP from the measured reflected birefringence, wherein

$$DoLP = \frac{\sqrt{{Q}^{2} + {U}^{2}}}{I} ;$$

2θ=tan⁻¹(U/Q);

$$DoVP = \frac{Q}{I} and DoHP = \frac{U}{I} ;$$

exposing the biological structure to a second light at a second polarization angle, which second angle is greater than the first angle; and, repeating steps a-e over a range of at least 5 degrees.

In other embodiments, the invention includes monitoring a change in birefringence status of a biological structure (which change corresponds with a structural or physical change in the biological structure due to a treatment). In such embodiments, a first angular dependence of a birefringence of the biological structure relative to an input polarization is determined as described above. The structure being monitored is then subjected to a treatment and a second angular dependence (again similar to above) is determined. The first angular dependence and the second angular dependence are then compared. Any differences between the two measurements can thus indicate a change in the birefringent status of the structure being monitored due to the treatment received.

In the various embodiments herein, the light to which the biological structure being monitored is exposed to, can also pass through one or more other biological structures. Such additional biological structures through which the light passes can optionally also be birefringent biological structures. For example, the biological structure being monitored can be, e.g., a tendon and the additional biological structure through which the light passes can be, e.g., skin layers such as epidermis, dermis, etc. In various embodiments wherein the light passes through an overlying structure to an underlying structure (again, e.g., a layer of skin collagen over an underlying tendon), both of the structures can optionally be monitored, e.g., for possible alteration due to treatment. In other embodiments, just one of the structures is monitored (e.g., either of them). Additionally, either one or both of the structures can optionally targeted for treatment. Additionally, either one or both of the structures can be monitored through determination of DoLP, θ, DoVP, DoHP, or the angular dependence of the birefringence of the biological structure(s) relative to an input polarization.

In the various embodiments herein, the biological structure(s) being monitored can comprise collagen structures. The structures can be one or more of: dermal collagen, mucosal collagen, synovial collagen, cartilage, a tendon, a ligament, a fascia, or an aponeourosis. In the various embodiments, the biological structure(s) can comprise, e.g., skin, a fascia, an aponeurosis, a tendon, a ligament, a capsule, a cartilaginous structure, a vascular wall, a vaginal wall, an introitus, a uterus, a urethra, a soft palate, a turbine, a gastrointestinal tract, including sphincters, or a structure of the airway.

Also, in the various embodiments comprising altering a polarization angle of the light to which the monitored structures are exposed, the degree to which the polarization angle is changed (e.g., in each determination of DoLP, etc.) can be, e.g., less than 1 degree, 1 degree, 2 degrees, 3 degrees, 4 degrees, 5 degrees, 6 degrees, 7 degrees, 8 degrees, 9 degrees, 10 degrees, 15 degrees, or 20 degrees or more greater than the first polarization angle. Thus, the increments over which birefringence is measured can be quite fine. Additionally, no matter the increments in which it is done, the range over which the birefringence is measured can be, e.g., 5 degrees or less, over 5 degrees, over 10 degrees, over 20 degrees, over 30 degrees, over 40 degrees, over 50 degrees, over 60 degrees, over 70 degrees, over 80 degrees, over 85 degrees, over 90 degrees, over 95 degrees, or over 100 degrees.

In the various methods herein (e.g., determination of DoLP, θ, DoVP, DoHP or the angular dependence of the birefringence of the biological structure(s) relative to an input polarization) the degree of linear polarization can be estimated from measurements other than Q, I, and U. Thus, in some embodiments, the measurement of reflected birefringence (from a biological structure) by a detection polarizer can be measured at +1 degree and +46 degrees, etc. Also, in some such embodiments, the reflected birefringence measurement by a detection polarizer can be measured at just 2 points (e.g., 0 and +45, etc.) or just 3 points rather than at 4 points (e.g., 0, −45, +45, 90).

In the various methods described herein the degree of polarization (DoP) is used as a general term to describe the combination of two or more measurements of light intensity. The two or more measurements can differ according to the states of the input (excitation) polarization, detection (emission) polarization, or the sample orientation. In some embodiments herein the methods are carried out by exposing a biological structure to light at a first input polarization state, with the structure at a first sample orientation state. Light reflected from the biological structure is then collected and detected at a first output polarization state. The input polarization state, output polarization state, or sample orientation state are changed to at least one additional state and at least one additional measurement of light reflected from the biological structure is performed. The degree of polarization is computed by combining at least two of these reflected light measurements at two different states. Finally, the degree of polarization is related to the degree of birefringence of the biological structure. In some embodiments herein, the degree of birefringence is then repeatedly measured in order to assess the effect of or provide feedback for one or more treatment methods.

In some embodiments herein, the methods are carried out by systems/devices that include a computer processor. Such computer processor comprises an instruction set to calculate, e.g., the DoLP, θ, DoVP, DoHP or the angular dependence of the birefringence of the biological structure(s) relative to an input polarization at particular polarization angles. The instruction set can include instructions to determine, e.g., the minimum or maximum DoLP, θ, DoVP, DoHP or the angular dependence of the birefringence of the biological structure(s) relative to an input polarization. In the various embodiments herein, the computer processor outputs its results (e.g., the calculations of DoLP, etc. at particular polarization angles, the minimum or maximum DoLP, etc.) to a user. The output can be, e.g., in printed form, an email or text message, displayed on a screen or monitor, etc.).

In certain embodiments herein, the methods can monitor the effect of any of a number of different treatments to a biological structure. Such treatments can include, but are not limited to, e.g., application of physical energy, application of radio frequency waves, application of ultrasound, application of heat, application of cold, or application of a cosmeceutical. In some embodiments, the treatment is passage of time.

In other embodiments of the methods herein, the light to which the biological structures is exposed (and the reflected light from the structures) is polarized light (e.g., linear polarized light, circularly polarized light, etc.). In some embodiments, the light to which the structure(s) are exposed (and optionally the light reflected back from the structure) is infra-red light, UV light, light of a wave length from about 800 to about 1100 nm, or fluorescence.

In some embodiments herein the invention comprises methods of monitoring a change in a biological structure in a tissue (e.g., a collagen structure). In such embodiments, the tissue is exposed to a first light; a first reflected light reflected from the structure is measured; the tissue is exposed (and/or the structure) is exposed to one or more treatments that can (or that can putatively) alter the physical characteristics of the structure; the tissue is exposed to a second light; a second reflected light reflected from the structure is measured; and the first reflected light and the second reflected light are compared in order to monitor any change in the structure. In such embodiments, the tissue can optionally include a second biological structure and both or either one of the two structures can be monitored for change induced by the treatment. The treatment can be directed to either one or both of the biological structures. However, even if the treatment is directed to only structure in particular (e.g., a tendon), it will be appreciated that an overlying structure (e.g., collagen layers in the skin) could possibly be impacted by the treatment and can therefore be monitored for change as well. In various embodiments, the two structures can be monitored simultaneously or sequentially. Furthermore, in some instances, one structure is closer to the source of the monitoring light and/or closer to the treatment application. In some embodiments, the structure can comprise, e.g., collagen, dermal collagen, mucosal collagen, synovial collagen, cartilage, a tendon, a ligament, a fascia, or an aponeourosis. The tissue can optionally include, e.g., skin, a fascia, an aponeurosis, a tendon, a ligament, a capsule, a cartilaginous structure, a vascular wall, a vaginal wall, an introitus, or a urethra. Also in such embodiments, the treatment can comprise one or more of, e.g., application of physical energy, application of radio frequency waves, application of ultrasound, application of heat, application of cold, or application of a cosmeceutical.

In other embodiments of the methods herein, the light to which the biological structures is exposed (and the reflected light from the structures) is polarized light (e.g., linear polarized light, circularly polarized light, etc.). Thus, in some embodiments, comparing the light comprises comparing the polarization of the light. In some embodiments, the light to which the structure(s) are exposed (and optionally the light reflected back from the structure) is infra-red light, UV light, light of a wave length from about 800 to about 1100 nm, or fluorescence. Also, in some embodiments of such methods, the comparison of the first and second reflected lights comprises determination of DoLP, θ, DoHP, DoVP or the angular dependence of the birefringence of the biological structure(s) relative to an input polarization.

In the various methods herein, the treatment that is monitored can comprise, e.g., application of physical energy, application of radio frequency waves, application of ultrasound, application of heat, application of cold, or application of a cosmeceutical. In some embodiments, the “treatment” is the passage of time (i.e., no additional modification such as thermotherapy is performed to the structure). The light applied to the tissues herein (e.g., via laser) can comprise infra red light, light of wavelength of about 800 to about 1100 nm, UV light, etc. Furthermore, in the methods herein, the light applied to the tissues to monitor status of structures (e.g., collagen) can comprise polarized light (e.g., linearly polarized, circularly polarized, etc.).

In various embodiments of the methods herein, e.g., when utilizing polarized light, the status and/or change in status of different collagen structures can be monitored through use of differently polarized light. For example, the status of one collagen layer (e.g., a dermal layer) is optionally monitored when the light exposed onto the tissue is at one polarization, while the status of another collagen layer (e.g., an underlying tendon) is optionally monitored when the light is at another polarization. In various embodiments, the polarization is switched back and forth between two or more polarization settings to monitor different collagen structures/layers in a tissue.

In the various embodiments herein, the information on collagen structure is gathered (i.e., the collagen is monitored) without invasion of the tissue or collagen structure or with only minimal invasion of the tissue/structure. Furthermore, the various collagen structures can be monitored through one or more layers of untargeted tissue (e.g., overlaying tissue and/or other collagen layers).

The information gathered by the methods and devices/systems of the invention can be used to, e.g., guide clinical decisions (including decisions concerning the continuation/cessation of treatment of the tissues; the effectiveness or lack thereof of the treatments; etc.). Thus, in various embodiments, treatment can be altered, e.g., discontinued when a percent change in measured collagen change is noted through, e.g., a percent change in polarization/birefringence of the light reflected from the structure. In other embodiments, treatment can be stopped when a certain desired result is reached in the structure being treated and/or when a certain percent change in the treated structure and/or in another ancillary structure (e.g., an overlaying dermal collagen layer) is reached. Again, such percent change is optionally indicated by a percent change in polarization/birefringence of the light reflected from the treated (or otherwise monitored) structure. In some embodiments herein, the methods comprise a feed-back control over treatment.

In the various embodiments herein, the methods can include steps for orienting the polarization of the light exposed on the tissue relative to the strand orientation of a collagen layer in the tissue (e.g., in parallel with the collagen orientation in a tendon, etc.).

In other embodiments, the methods include the use of a plurality of source-detector distances in differentiating between changes in various collagen structures. Also, the methods include wherein wavelength dependence of birefringence is measured for various collagen structures.

In yet other aspects, the invention includes methods of determining the collagen content in a tissue wherein a measurement of birefringence provides an estimation of the collagen content. Furthermore, the invention also includes embodiments comprising methods of monitoring collagen status through measurement of birefringence which, in turn, is used as a guidance for treatment of the collagen layer being treated and/or of ancillary/nearby tissues/structures such as other collagen structures.

In other embodiments herein, the invention comprises a system or device for monitoring a change in one or more structures in a tissue (e.g., collagen structures). Such systems can include: a light source component (configured to emit light to the tissue); one or more light polarizer components (configured to polarize light that is emitted from the light source or used for light reflected back from the tissue and to be set at particular angles of φ); one or more lens components (configured to focus light that is emitted from the light source or to focus light that is reflected from the tissue); a light detection component that is configured to detect light reflected from the tissue (e.g., light that has also optionally traveled through other components such as lens(es), polarizers, etc.); a lock-in amplifier component that is configured to amply the light reflected from the tissue; and, a computer or processor component which has an instruction set that is programmed to instruct one or more of: direct the light source to expose the tissue to a first light at a particular polarization; direct the detection component to measure the polarization of a first reflected light from the one or more structures; direct the light source to expose the tissue to a second light at a particular polarization; direct the detection component to measure the polarization of a second reflected light for the one or more structures; and, compare the first reflected light and the second reflected light, thereby monitoring the changes in the one or more structures (based on changes in the polarization of the light) and to output the results to a user (e.g., on a monitor or readout, on a printout, on a disc or other medium, etc.). In many embodiments, the computer component is programmed to direct the emission and detection of the second light after the tissue that comprises the structure has been exposed to a treatment (e.g., RF treatment, exposure to a cosmeceutical, application of physical energy, application of radio frequency waves, application of ultrasound, application of heat, application of cold, etc.). In some instances, the “treatment” can merely be the passage of time rather than application of a particular therapy or the like.

In other embodiments herein, the invention comprises a system or device for monitoring the degree of birefringence in one or more structures in a tissue (e.g., collagen structures). Such systems can include: a light source component (configured to emit light to the tissue); one or more light polarizer components (configured to polarize light that is emitted from the light source or used for light reflected back from the tissue and to be set at particular angles or used to rotate the polarization of light prior to sample excitation or prior to emission detection); a light detection component that is configured to detect light reflected from the tissue (e.g., light that has also optionally traveled through other components such as polarizers, polarization rotators, etc.); and, a computer or processor component which has an instruction set that is programmed to instruct one or more of: direct the detection component to measure a first reflected light at a particular polarization state from the one or more structures following exposure of the light source to the tissue to light at a particular polarization state; direct the detection component to measure additional reflected light at a particular polarization state from the one or more structures following exposure of the light source to the tissue to light at particular polarization state; and, compare the first reflected light and the additional reflected light, thereby monitoring the degree of birefringence in the one or more structures and to output the results to a user (e.g., on a monitor or readout, on a printout, on a disc or other medium, etc.). In many embodiments, the computer component is programmed to direct additional measurement of the degree of birefringence after or while the tissue that comprises the structure is exposed to a treatment (e.g., RF treatment, exposure to a cosmeceutical, application of physical energy, application of radio frequency waves, application of ultrasound, application of heat, application of cold, etc.). In some instances, the “treatment” can merely be the passage of time rather than application of a particular therapy or the like.

The computer can also be programmed to control one or more of the various components present in the various embodiments of the invention. Thus, the computer can optionally control, e.g., the intensity of the light emitted, the timing and duration of the light emitted, the degree of polarization of the light emitted to the tissue, the angle setting of the detection polarizer through which birefringent light is transmitted to the detector, etc. In some embodiments, the system/device of the invention comprises multiple polarizer components, e.g., optionally in the light path prior to tissue exposure (e.g., light polarizer components that polarize light from the light source prior to the light exposing the tissue) and/or optionally in the light path of the light reflected back from the tissue towards the detector (e.g., light polarizer components that are set at preset angles such as +45 degrees, −45 degrees, 0 degrees, 90 degrees, etc.) The polarizer components can be directed/controlled by one or more polarizer rotators (which in turn can be optionally controlled by instructions either directly from the user or from instructions from the computer component which can also be input from the user). In some embodiments, the systems/devices of the invention can comprise multiple lenses. Thus, in some embodiments, the systems/devices can optionally have a lens that focuses light from the light source prior to the light exposing the tissue and/or can optionally have a lens that focuses the light that is reflected back from the tissue. In some embodiments, the systems/devices can comprise one or more mirrors (e.g., to direct light to and/or from the light source, the tissue, etc.). Of course, particular embodiments herein do not comprise mirrors. Some embodiments also comprise one or more polarization compensator components (e.g., that are each operably connected to a polarizer component). In the various embodiments, the system or device of the invention can be used to monitor tissues having a first and at least a second collagen structure. In such embodiments, the computer component can be programmed to direct the light source and polarization components to expose the tissue to a first light at a first polarization orientation and a second light at a second polarization orientation. Furthermore, in such embodiments, the computer component is programmed to differentiate changes in the first collagen structure from changes in polarization of the first light and changes in the second collagen structure from changes in polarization of the second light. In various embodiments, the systems/devices of the invention monitor a change in one or more structures in a tissue (e.g., due to treatment of the tissue). Such change can be, e.g., a change in DoLP, a change in θ, a change in DoHP, a change in DoVP, or a change in the angular dependence of the birefringence of the biological structure(s) relative to an input polarization.

In some embodiments, the systems or devices or methods herein comprise instrument calibration or correction to improve the accuracy of the measurement of the degree of birefringence. Such calibrations or corrections may be determined in advance of the measurement of the sample of interest (e.g. biological structures), and may employ calibration standards (e.g. mirrors, Teflon, or other reflective materials). The calibration may involve additive or multiplicative correction or a combination of the two. In some embodiments, multiple measurements made on the sample of interest are used to self-calibrate or self-correct the determined degree of birefringence.

In particular embodiments, the systems herein can comprise systems such as (or similar to) the ones illustrated in FIG. 1, 2, 3, 7, 13, 16, or 17 (and as described in the corresponding areas of the specification). Some such embodiments will not comprise mirrors (M) as shown in the Figures and/or will not comprise sample stages/platforms. In various embodiments of the systems/devices, the components that typically interact with light (either directly or through control of other components) before the light is exposed to a tissue can include (but are necessarily limited to) one or more of: a light source; a polarizer a polarization rotator (liquid crystal); a polarization compensator; a focusing lens; a fiber optic; a polarization controller, a polarization rotation controller; and a computer/processor. Also, in various embodiments, the components that can typically interact with light (either directly or through control of other components) after the light is reflected back from a tissue can include (but are necessarily limited to) one or more of: a parabolic mirror; a polarizer; a lens (e.g., a detection lens or a collimating lens); a detector; a sampling lens; a lock-in amplifier; a polarization compensator; a polarization rotator; a fiber optic; a polarization controller; a trans-impedance amplifier; and a computer/processor. It will be appreciated that computer/processor components can optionally control any or all of such components, e.g., in terms of usage and/or settings, and can optionally output any parameters set or measured for each component (e.g., polarization angles, light intensity, etc.) to a user. The various components of the systems herein are typically operably connected to at least one other component in the system of which such component is a part.

These and other features of the invention will become more fully apparent when the following detailed description is read in conjunction with the accompanying figures and claims.

### BRIEF DESCRIPTION OF THE DRAWINGS

FIG. 1 presents a schematic of an example configuration of system components of an embodiment of the invention.

FIG. 2 presents a schematic of an example configuration of system components of an embodiment of the invention.

FIG. 3 presents a schematic of an example configuration of system components of an embodiment of the invention.

FIG. 4 presents a graph illustrating birefringence of various substrates monitored through an embodiment of the invention.

FIG. 5 presents a graph illustrating birefringence of a half wave plate with mirror monitored through an embodiment of the invention determining DoLP.

FIG. 6 presents a graph illustrating birefringence of a half wave plate with minor monitored through an embodiment of the invention determining amount of polarization rotation.

FIG. 7 presents a schematic of an example configuration of system components of an embodiment of the invention.

FIG. 8 presents a graph illustrating birefringence of bovine tendon monitored through an embodiment of the invention.

FIG. 9 presents a graph illustrating birefringence of bovine tendon measured through a gelatin layer monitored through an embodiment of the invention.

FIG. 10 presents a graph illustrating birefringence of bovine tendon measured through a Teflon layer monitored through an embodiment of the invention.

FIG. 11 presents a graph illustrating birefringence of bovine tendon measured through an Intralipid layer monitored through an embodiment of the invention.

FIG. 12 presents a graph illustrating birefringence of porcine skin monitored through an embodiment of the invention.

FIG. 13 presents a schematic of an example configuration of system components of an embodiment of the invention.

FIG. 14 presents a flowchart outlining monitoring steps in an example embodiment of the invention.

FIG. 15, Panels A and B, presents a flowchart outlining monitoring steps in an example embodiment of the invention.

FIG. 16 presents a schematic of an example configuration of optical components of the invention positioned in relation to a tissue surface.

FIG. 17 presents a schematic of an example configuration of electrical components of an embodiment of the invention.

FIG. 18 presents a schematic of an example sampling lens of an embodiment of the invention.

FIG. 19 presents a schematic of an example configuration of system components of an embodiment of the invention.

FIG. 20 presents a graph illustrating birefringence of porcine skin, measured at particular sample orientation (γ=0), monitored through an embodiment of the invention.

FIG. 21 presents a graph illustrating birefringence of porcine skin, measured at another particular sample orientation (γ=45), monitored through an embodiment of the invention.

FIG. 22 presents a graph illustrating birefringence of porcine skin, computed from measurements made at 2 sample orientations (γ=0 and 45), monitored through an embodiment of the invention.

FIG. 23 presents a graph illustrating birefringence of live human skin, computed from measurements made at 2 sample orientations (γ=0 and 45), monitored through an embodiment of the invention.

FIG. 24 presents a graph illustrating birefringence of human in vivo forearm skin as a function of thermal treatment, monitored through an embodiment of the invention.

FIG. 25 presents a schematic of an example configuration of a calibration standard of an embodiment of the invention.

### DETAILED DESCRIPTION

The ability to accurately monitor (and thereby more accurately control) tissue treatments such as thermotherapy, especially in real time, is significant in the productive treatment of a number of disease states/medical conditions, e.g., treatment of joint trauma. Furthermore, the ability to accurately monitor the effect of such products as cosmeceuticals on tissues (whether during and/or after treatment/application of such) is quite significant. Various embodiments of the current invention utilize tracking of changes of reflected light from biological structures, or from multiple biological structures, to track corresponding changes in such structures arising from treatment. Of course, it will be appreciated that even though the various embodiments herein are primarily described as useful for tracking progress of tissue treatment and the like, that the benefits of the invention also extend to, e.g., monitoring the condition of a subject's tissue or the presence and/or progression of a disease state or medical condition, whether or not any treatment is administered.

In particular embodiments herein, the invention uses birefringence of light reflected from one or more layers of collagen tissue to monitor treatment progress on collagen. Optionally such changes are tracked in real time, e.g., during treatment of the tissue. In yet other embodiments, the invention uses optical monitoring procedures other than birefringence and/or tracks changes after treatment has occurred (e.g., rather than as treatment is occurring). The invention includes the methods of monitoring treatment effects as well as systems and devices that implement such methods. Overall, the invention results in increased monitoring ability for tracking of tissue treatment.

### DEFINITIONS

Before describing the present invention in detail, it is to be understood that the invention herein is not necessarily limited to use with particular light sources, thermotherapy treatments, etc., which can, of course, vary. It is also to be understood that the terminology used herein is for the purpose of describing particular embodiments only, and is not necessarily intended to be limiting. As used in this specification and the appended claims, the singular forms “a,” “an,” and “the” include plural referents unless the context clearly dictates otherwise. Thus, for example, reference to “a laser” optionally includes a combination of two or more lasers, and the like.

The term “subject” as used herein includes, but is not limited to, a mammal, including, e.g., a human, non-human primate (e.g., monkey), mouse, pig, cow, goat, rabbit, rat, guinea pig, hamster, horse, monkey, sheep, or other non-human mammal, or a non-mammal, including, e.g., a non-mammalian vertebrate, such as a bird, reptile, or amphibian. In some embodiments, the methods and systems/devices of the invention are used to monitor non-human animals. Many commercially important animals are susceptible to medical conditions, e.g., joint trauma, whose treatment is optionally monitored with the current invention.

The term “footprint” as used herein refers to the tissue area monitored by the current invention. It will be appreciated that such area can vary in different embodiments depending on, e.g., size of illumination beam used, etc. Furthermore, it will also be appreciated that such monitoring footprint need not be, and often is not, the same size as a treatment footprint, i.e., a tissue area that is being treated by, e.g., thermotherapy, etc.

As will be appreciated, the above terms, as well as additional terms, are detailed/described further below.

### Real-Time Monitoring of Tissue Treatment

In the various embodiments of the present invention, changes in biological structures (e.g., collagen structures) are monitored by directing light into a tissue and collecting the light after it has interacted with the structures within the tissue. In particular embodiments, the collected light is measured in order to monitor the status of and/or changes in the structures within the tissue. In the various embodiments herein, more than one structure can be monitored simultaneously or sequentially, e.g., during the course of a treatment process that involves the tissue or during the course of the progression of a disease state or medical condition.

Particular embodiments of the invention monitor, through exposure to light, the status and/or change in status of collagen. As discussed further below, collagen is a uniaxial birefringent material whose optic axis (or slow axis), in which direction light travels most slowly, is parallel to the long axis of its triple helix while its fast axis, the one in which direction light travels most quickly, is perpendicular to its triple helix axis. The difference in refractive index, Δn, between the slow and fast axes of collagen is approximately 3×10⁻³. See, e.g., D. J. Maitland and J. T. Walsh, “Quantitative Measurements of Linear Birefringence During Heating of Native Collagen” Lasers in Surgery and Medicine, 20:310-318 (1997). The phase shift (δ) introduced between light traveling along the slow vs. fast axes can be related to the thickness of the collagen sample (d) and the wavelength of light (λ) according to Equation 1.

$$δ = \frac{2 π d Δ n}{λ} \qquad ( Equation 1 )$$

Linearly polarized light that is oriented parallel to the collagen optic axis (hereafter referred to as 0 degree orientation) will undergo no polarization rotation upon interaction with the collagen. However, linearly polarized light at a 45 degree orientation will undergo maximal polarization rotation. According to Equation 1, linearly polarized light with 800 nm wavelength and 45 degree orientation relative to the optic axis will undergo a 90 degree rotation (or 180 degree phase shift) after traveling through only 130 μm of collagen. Collagen layers in humans can vary in thickness depending on their type and location. For example, collagen layers in skin can be ˜200 microns in thickness, while tendons can be ˜2-3 mm in thickness. As explained below, various embodiments of the invention can monitor at various depths. Thus, some embodiments of the invention can monitor, e.g., 1-2 mm within a tendon, etc.

Again, as discussed further below, the birefringence of collagen is lost upon denaturation. Therefore, the embodiments herein can monitor changes in birefringence of a tissue (e.g., before and after thermotherapy) in order to track the progression of collagen denaturation by heating. Thus, for example, a tendon can be monitored before treatment and the birefringence thus determined can be compared to the birefringence determined after (or during) treatment to thereby monitor changes if any in the tendon (such as denaturation of its collagen). See Maitland, supra.

Various embodiments of the invention utilize the change in birefringence due to denaturation to monitor, e.g., the progress or effectiveness of treatments and the like. In particular embodiments, the baseline or starting status birefringence of a structure (e.g., a collagen layer) and the structure's response to treatment are both monitored by directing, e.g., a linearly polarized laser light into the tissue which comprises the structure, collecting the light after it has interacted with the structure(s), and measuring polarization-dependent properties of the collected light (e.g., the extent or degree of depolarization, the amount of polarization rotation, etc.). In various applications, the starting status of the structure is typically the status before any treatment is applied to it. However, the starting status can also optionally be from a point after treatment has started. Thus, for example, monitoring can optionally be implemented in the middle of a course of treatment of a tissue and be used to track changes occurring after the start of monitoring.

In various embodiments, the monitoring can be done non-invasively (e.g., by directing light upon skin, etc.), while in other embodiments, the monitoring can involve an invasive act (e.g., a monitoring probe or component inserted (e.g., arthroscopically) into a subject to monitor a tendon, vessel wall, etc.).

In some embodiments, the invention can be used to determine collagen content in a tissue (e.g., whether or not any treatment has been or is to be administered). For example, light properties such as birefringence can be measured in multiple subjects and/or at multiple sites within a subject to create a measurement guide of collagen content/status based on the light property measured (i.e., as opposed to changes in such property used in some embodiments herein). Based on multiple readings (between the level of the light property measured and collagen content), such measurement guide thus allows a practitioner to measure or estimate the collagen level in a tissue. The measure/estimate of collagen (based on the light property measured) can be done prior to any treatment to the subject or to compare with an average measurement (e.g., as in comparing diseased tissue against non-diseased tissue, etc.). Thus, a practitioner can use such measurement to advise whether treatment should even be undertaken, whether or to what extent treatment may be successful, etc. The readings taken to construct the measurement guide can optionally be normalized for subject status (e.g., based on age, ethnicity, gender, etc.) and tissue type or location (e.g., dermal collagen in the face, dermal collagen in the hands, etc.).

In certain embodiments, e.g., those comprising linearly polarized laser light, the light can be used to distinguish between birefringence from a superficial layer of collagen (e.g. an epidermal, dermal, mucosal, synovial, or intimal structure) and a deeper layer in which collagen is substantially oriented in linear strands (e.g. tendons, ligaments, fascia, aponeurosis, etc.). In some embodiments, one layer of collagen is targeted for treatment while the other is anticipated to remain untreated. However, it will be appreciated that both layers can be targeted for treatment. In various embodiments, the structures targeted for treatment as well as the structures not targeted for treatment can be monitored by embodiments of the current invention. Of course, in some embodiments, the invention monitors only a single structure rather than multiple structures (e.g., a tendon rather than a tendon and an overlying dermal collagen layer) even if such monitoring of a single structure is done through another structure (e.g., monitoring of a tendon or fascia underneath skin, etc.).

It will be appreciated, that while for ease of description, the current description herein primarily describes the structure type that is monitored as collagen, other structures are optionally included in the various embodiments. Thus, it is contemplated that the invention can also find use with monitoring of, e.g., pathological tissue such as tumors that are treated with thermotherapy. In such embodiments, it is thought that the methods and systems of the invention will track such tissues via, e.g., birefringence or other optical methods such as fluorescence. Furthermore, in some embodiments the structure monitored can comprise keratin and/or elastin. Additionally, while particular light (e.g., linearly polarized laser light) and light properties (e.g., changes in birefringence, etc.) are detailed in the discussion herein, those of skill in the art will appreciated that other light types, and/or light-dependent properties are included within the spirit and purview of the application and scope of the appended claims. Thus, different light properties can be measured rather than polarization-dependent properties. Additionally, the light used to monitor changes in the structure(s) can be, e.g., non-polarized, linearly polarized, or circularly polarized light. Furthermore, the light used can be, e.g., at a single wavelength, i.e., monochromatic (or substantially monochromatic), or at a multiplicity of wavelengths. In embodiments comprising use of polarization-dependent properties, such properties can include, e.g., the difference between the absorbed intensity of two light beams having mutually perpendicular linear polarizations, the difference between the absorbed intensity of two light beams having left and right circular polarizations, the rotation of the polarization of a linearly polarized input light beam, the extent of depolarization of a polarized light bean, or the polarization ellipticity of a circularly polarized input light beam.

### Exemplary Uses of the Methods/Devices

In various embodiments, the methods of the invention comprise placement and orientation of the various system components (e.g., light emitter and detector) in relation to the tissue/structure being monitored. As will be appreciated, such placement/orientation can involve movement of the tissue being monitored and/or movement of one or more components of the devices/systems herein. Also, while the various embodiments herein are primarily discussed in terms of generalized systems of components, particular embodiments can comprise self-contained devices (e.g., a handheld probe and/or a handheld probe operationally connected to a unit having laser components, etc.). See below. Furthermore, as mentioned throughout, the various methods of the invention and the various devices/systems of the invention can be used topically on subjects (i.e., noninvasively) and/or can be used internally within subjects (i.e., invasively either through incisions or the like or through orifices of the subjects).

By determining a baseline status measurement of a tissue structure's birefringence, the various embodiments of the invention allow comparison of such values with measurements taken after/during a treatment (or even taken at a later date) to establish the impact (if any) of a given intervention. Thus, the monitoring can be real time during the treatment and/or after the treatment. Additionally, in some embodiments by simultaneously monitoring birefringence at multiple polarization settings while delivering a treatment (e.g. radiofrequency, ultrasound, light) the treatment can be terminated when either the target structure (e.g., a tendon) or a non-target or secondary target structure (e.g., a superficial layer such as dermal, synovial, mucosal collagen, etc.) has reached the desired change in birefringence or has exceeded a threshold change in birefringence, in either instance indicating a change in the particular structure. Thus, through use of some embodiments, a particular collagen containing structure can be treated while simultaneously avoiding damage to other collagen containing structures located above or below the targeted structure.

Also, it will be appreciated that various embodiments of the invention herein can include systems having multiple light sources and detector/polarizer component paths as well as systems having adjustable light sources and detection/polarizer component paths to allow monitoring of multiple tissue structures and/or tissue structures in multiple locations or depths.

One step in various methods herein includes the determination of birefringence of the tissue being monitored. The determination of the tissue birefringence can be accomplished by the devices/systems of the invention in several ways. Many of the embodiments of the invention can be grouped into one of two general categories: embodiments using the extent of depolarization of a polarized light beam in order to determine tissue (e.g., collagen) orientation and embodiments using the amount of polarization rotation of a polarized light beam in order to determine tissue orientation. However, it should be appreciated that the various embodiments herein typically share many qualities and aspects. For example, the various embodiments herein can, unless stated otherwise, all use similar light sources, wave plates, polarizers and other components. See below. Also, each general classification group of embodiments comprises specific embodiments wherein multiple tissue layers can be monitored simultaneously or concurrently and specific embodiments wherein only a single tissue is monitored (however, optionally through one or more other tissues). Other areas of similarities will be apparent to those of skill in the art.

### Degree of Linear Polarization as Function of Collagen Axis Orientation

As stated above, one class of embodiments herein comprises determination of tissue orientation (by monitoring of which any change such as denaturation can be tracked) through determination of the extent or degree of depolarization of a polarized light by the tissue. Thus, in some embodiments, the methods and devices of the invention determine the degree of linear polarization of light reflected from a tissue (e.g., a tendon of collagen) as a function of the orientation of the tissue (e.g., the orientation of the axis of the collagen). Light diffusely reflected from tissue such as collagen travels through a wide range of possible path lengths when it is reflected. As can be seen from equation 1 above, the effect of polarized light transmitted at a 45 degree orientation from the axis of orientation of a tissue traveling through a range of depths of the tissue will be polarization rotation over a wide range of angles. Since only about 130 μm of collagen is required to induce a 90 degree change in polarization rotation (at a wavelength of 800 nm), light scattered from a thicker slab of collagen (e.g. 1 mm) will undergo a mixture of all possible polarization rotations. Thus, when monitored, the integrated result seen by a detector will be a large reduction in the degree of linear polarization. However, at 0 degree orientation between the polarized light and the tissue axis, there is no birefringent effect. Therefore, at this 0 degree orientation, polarized light can be expected to be less depolarized within the tissue. These effects can be seen in polarized images of rat tails. See, e.g., P. J. Wu, and J. T. Walsh, Jr., “Stokes Polarimetry Imaging of Rat Tail Tissue in a Turbid Medium”, J. Biomed. Optics, 11 (2006).

Therefore, in some embodiments, the methods and devices of the invention utilize the following method of searching for the tendon (or other tissue) orientation: search for the minimum (or maximum) degree of linear polarization as a function of sample orientation. The degree of linear polarization (DoLP) is defined as:

$$\begin{matrix}DoLP = \frac{\sqrt{{Q}^{2} + {U}^{2}}}{I} & ( Equation 2 ) \\ where \text{:} & \\ Q = {I}_{0} - {I}_{90} & ( Equation 3 ) \\ U = {I}_{+ 45} - {I}_{- 45} & ( Equation 4 ) \\ I = {I}_{0} + {I}_{90} & ( Equation 5 )\end{matrix}$$

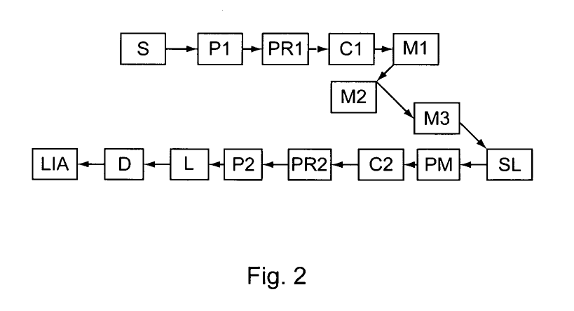

and I_{φ} is the signal measured on the detector when the detection polarizer (e.g., P2 in FIG. 2) is at an angle of φ. The angle at which the minimum DoLP is observed should thus correspond to an orientation angle of 45 degrees (or more generally: 45°±n*90°, where n=0, 1, 2 . . . ).

The above-defined quantities, Q, U, and I, are 3 of the 4 values, collectively referred to as the “Stokes vector,” (see, e.g., Kliger, et al., “Polarized Light in Optics and Spectroscopy,” Academic Press (New York), 1990) that completely define the polarization state of a light beam (the fourth value is the difference in intensity between left and right circularized light). Q and U can also be used to compute the linear polarization angle, θ.

2θ tan⁻¹(U/Q) (Equation 6)

Thus, in some embodiments, the linear polarization angle is determined through the methods and devices herein as a way of determining the polarization rotation induced by a birefringent tissue sample and thus the tissue's orientation. Equation 6 provides a way of estimating the polarization rotation. It should be noted that using equations 2 and 6, the same data (Q and U) can equivalently be used to compute either DoLP or θ, and either could be used to determine the birefringent axis of collagen.

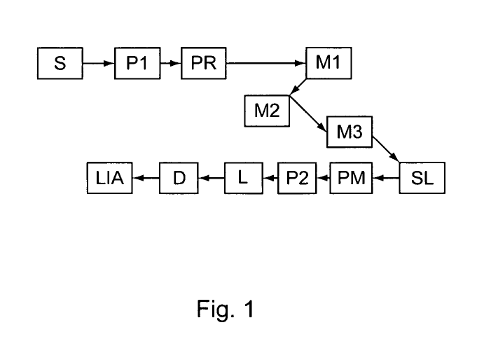

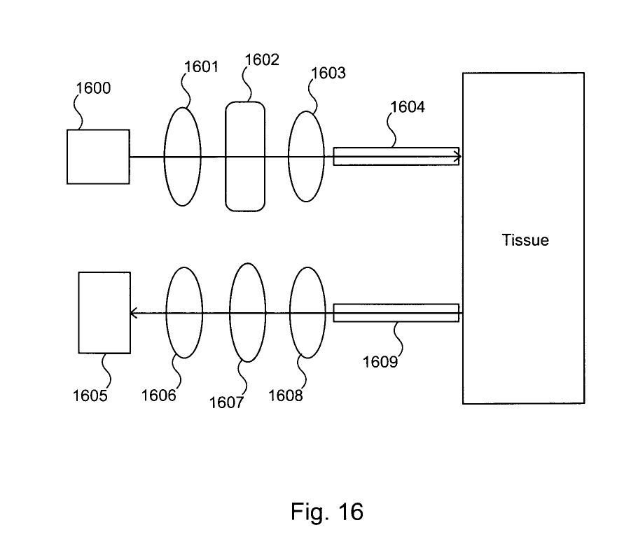

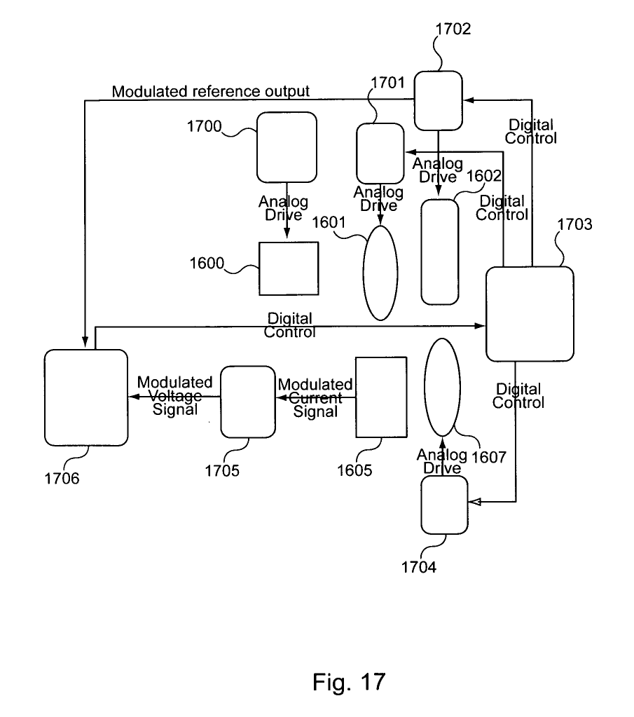

An exemplary arrangement of system components to implement such equations is shown in FIG. 2. In some embodiments, such an arrangement (as well as optionally other arrangements and optionally along with other components such as computer/processor components as in FIG. 17) can be used to determine the degree of linear polarization and/or the linear polarization angle. Once the values are determined, embodiments of the invention can monitor the tissue before, during, or after any treatment or to track tissue status over time without treatment, etc. As can be seen, the component arrangement outlined in FIG. 2 includes a number of components. For example, the arrangement includes polarization compensator plate C1 and compensator plate C2. Such polarization compensators (e.g., quarter-wave and/or half-wave plates) can correct for the depolarization induced by the optical elements in the apparatus (minors, lenses, etc) (inherent depolarization). Those of skill in the art will be familiar with compensators and their use and calibration. FIG. 2 also includes detector D (silicon photodiode with built-in trans-impedance amplifier); lens L (1″ diam. 75 mm FL); lock-in amplifier LIA (reference to source modulation); mirror M1 (0.5″ diam. round); mirror M2 (0.5″ diam. round); mirror M3 (1″ diam., D-shaped). Also, as can be seen, such arrangement can include polarizer P1 (source polarizer) to “clean-up” the polarization of the laser and polarization rotator PR1 (liquid crystal) to rotate the beam. The arrangement can also include second polarization rotator PR2 (liquid crystal) to allow for the automated measurement of the Stokes vector components. The configuration of mirrors in FIG. 2 (and in FIGS. 1-3, 7, and 13) is arranged to conveniently move the light from a horizontal to a vertical orientation to go up into the sample well used in the Examples. Thus, it will be appreciated that the arrangement of mirrors, and even the presence of mirrors, is optional and should not be taken as limiting. Cf. FIG. 16. In some embodiments comprising fiber optic transmission of light, mirrors would not be present. The arrangement in FIG. 2 also shows parabolic mirror PM (90 degree off-axis) used to take light from the sampling lens (in a vertical direction) and launch it back to horizontal. Again, the presence of such mirror in the illustrating examples herein should not be taken as limiting and it will be appreciated that other embodiments herein do not comprise such a mirror. FIG. 2 also includes polarizer P2 (source polarizer) and laser source S (808 nm, 200 mW, 1 kHz, square-wave modulated) and sampling lens SL (a split lens, 1″ diam. PCX, 15 mm FL, 1 mm black ABS spacer).

Thus, in some embodiments, the methods and devices of the invention can determine, e.g., the orientation of the birefringent axis of a tendon by:
a.) setting the appropriate voltage on PR2 (as in FIG. 2) and measuring I₋₄₅, I₀, I₊₄₅, I₉₀;
b.) computing Q, U, and I;
c.) computing DoLP and/or θ,
d.) changing the voltage on PR1 so that the input polarization is rotated by a small increment;
e.) repeating steps a-d until the PR1 setting required to achieve minimum DoLP has been located or repeating the steps until the maximum absolute value of θ is found;
f.) recording the PR1 setting from step e as the 45 degree orientation setting.
   Thus, in various embodiments, a user can set PR1 to a particular angle (either at random or based on visual or otherwise (e.g., ultrasound) determination of the orientation of the tissue being monitored) and monitor birefringence (via the LIA) at each of the four different angles of PR2 (see Equations 2-5). Such four values will give the measure of degree of polarization at that point. The angle of PR1 can then be reset and the process repeated. PR1 can be reset in small increments such as 5-10 degrees and the measurements taken over, e.g., ˜90-100 degrees in order to find the minimum and maximum for the degree of polarization of the sample. In the case of the liquid crystal polarization rotators such as are used in various embodiments, an appropriate voltage can be in the range of 0 to 5V, which corresponds to polarization rotations in the range of 0 to 180 degrees. Thus, a small incremental change would be the voltage change required to rotate the polarization by a few degrees or less. This would be a voltage change in the mV range. Similar steps in determining minimum/maximum birefringence are given below in the embodiments using determination of amount of polarization rotation.

Example 1 below, illustrates the determination of the axis of orientation of a tendon using the above.

As detailed above, various embodiments of the invention present methods and devices to detect and monitor collagen orientation and denaturation. Additional embodiments of the invention present automation and instrumentation to carry out such detecting and monitoring. For example, results of use of one such exemplary embodiment are described in Example 2. Furthermore, Example 3 shows the results of bovine tendon measured through various skin-like (“phantom”) materials. These embodiments illustrate the increasing penetration depth through which tendon birefringence can be measured by various embodiments of the invention.

As can be seen from Example 2, various embodiments of the invention comprise systems (e.g., comprised of light sources, detectors, polarization manipulators, computers/processors, etc.) that can automatically carry out the various methods of the invention (e.g., of determination of collagen strand orientation and changes in such thus indicating degree of denaturation of such, etc.). Of course, it will be appreciated that the precise system configuration in Example 2 should not necessarily be taken as limiting and that other embodiments can optionally comprise additional, alternative, or fewer system components depending upon the specific needs of the embodiment. Also it should be noted that while the graphs for Example 2 are presented in terms of sample orientation, they could also be presented in terms of PR1 orientation. Thus, the graphs represent the relative angle between the input linear polarization and the sample orientation.

In addition to illustrating an exemplary automated system of the invention, Example 2 also shows that embodiments of the invention can monitor the decrease in birefringence of a tissue (e.g., a tendon) due to denaturation.

### Tendon Measurements Through “Phantom” Skin Layers

As illustrated in Example 3, various embodiments of the invention can monitor tissue layers (e.g., collagen in tendons) through a number of various intervening layers (e.g., skin). Example 3 demonstrates that various embodiments of the invention can optionally be used for a number of non-invasive procedures such as tendon measurement through an intervening layer.

As shown in Example 3, in some embodiments, the methods and devices of the invention can split the measurement of the Degree of Linear Polarization (DoLP) into 2 parts: the Degree of Vertical Polarization (DoVP) and the Degree of Horizontal Polarization (DoHP). Such split terms can be defined as:

$$\begin{matrix}DoVP = \frac{Q}{I} & Equation 7 \\ DoHP = \frac{U}{I} & Equation 8\end{matrix}$$

where Q, U, and I are as defined above. Of course, it will be appreciated that other embodiments which include determination of degree of linear polarization can also use DoVP and DoHP (e.g., the determinations in Examples 1, 2, and 4 could have made calculations of DoVP/DoLP, etc.). Measurement of DoHP and DoVP as a function of angle is thought to result in curves having the same amplitude but offset in phase by 90 degrees. Splitting the DoLP into its horizontal and vertical components can optionally add in reliability when determining the amplitude of the change in the degree of polarization with sample orientation. This is particularly true if the DoVP and DoHP functions are simultaneously fit to a model function where certain parameters are held fixed, such as: (1) the relative amplitudes of the fitting function, and (2) the relative phases of the fitting function.

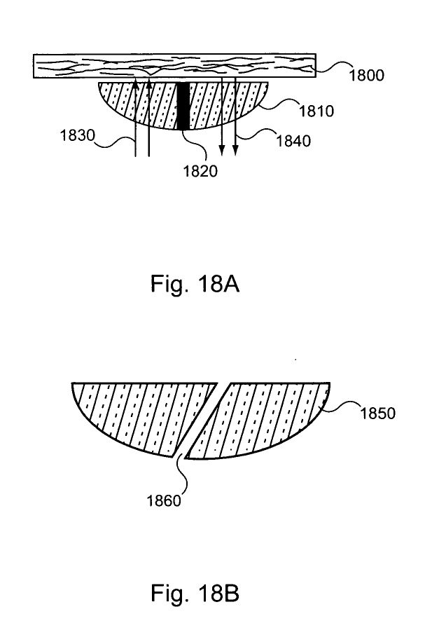

In various embodiments (optionally both those monitoring degree of polarization and those monitoring amount of polarization rotation) the methods and systems of the invention can comprise monitoring at various depths into the tissue being examined. For example, a split sampling lens (see FIG. 2 and FIG. 18) can optionally be used to control the monitoring depth. In such lenses, a spacer such as black ABS plastic (which does not transmit light) is placed between two halves of the lens. In FIG. 18, Panel A, split lens 1810 is shown next to tissue 1800, spacer (e.g., black ABS) 1820 is shown along with light 1830 entering the lens and light 1840 exiting the lens (from the tissue). Light can be transmitted through one half of the lens to a sample to be monitored and the resulting reflected light detected as it comes back through the other half of the lens. The thickness of the spacer can be used to control the monitoring depth into the sample. The bigger (thicker) the spacer is, the greater the depth that is measured. In other embodiments, the split lens comprises a “drilled lens” wherein a hole is drilled through the lens. The hole is then lined with a coating or inserted tube (such as ABS or similar). In FIG. 18, Panel B, a cross section of drilled lens 1850 is shown. Lined hole 1860 is also depicted. In such embodiments, the light is sent into the tissue through the hole and the remainder of the lens can be used for collection of the light reflected back from the tissue. Also in such embodiments, the thickness of the lining of the hole determines the depth of the light monitoring (similar to the spacer above). Such drilled lens embodiments can comprise an offset hole. See FIG. 18. It will be appreciated that a similar concept (changing depth monitoring) is outlined below in regard to the spacing distance between two optical fibers (input and output). Also, similar control over the monitoring depth can optionally be achieved through manipulation of the distance between the sample and the sampling lens and/or distance between the light source into the sample and detector out of the sample. Again, it will be appreciated that any of the embodiments herein can optionally comprise any necessary arrangement/component in order to manipulate the depth of penetration in the embodiments herein. Those of skill in the art will be familiar with various methods and arrangements to manipulate monitoring depth, e.g., so that the light source and cone of detection overlap appropriately at the desired depth.

### Measurement of Dermal Collagen Through an Epidermis Layer

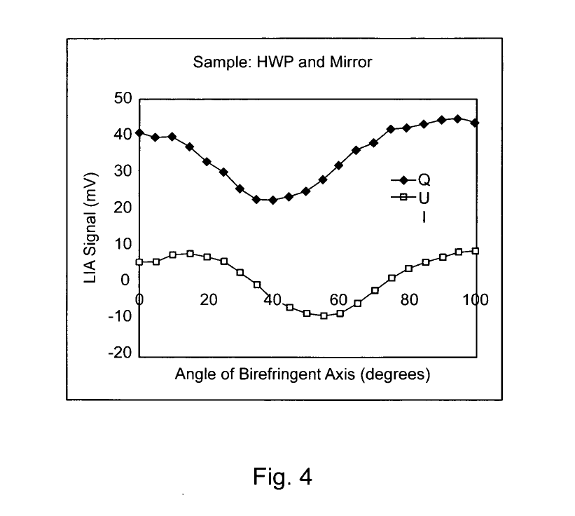

FIG. 4 illustrates that embodiments of the invention can be used to monitor the birefringence of a dermal collagen layer in a skin sample through illumination through an epidermis layer. It will be appreciated that collagen in skin (e.g., dermis) is less linearly oriented than that of tendons and thus the amplitude and/or shape of the angular variation of degree of polarization is optionally different in skin monitoring as compared to tendon monitoring.

### Automated Sample/Polarization Orientation

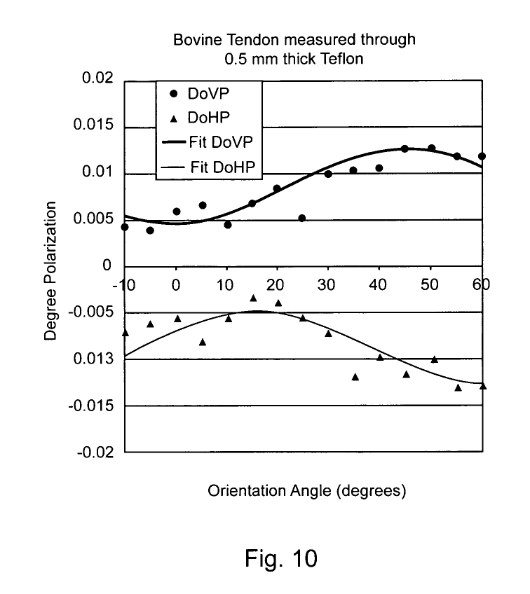

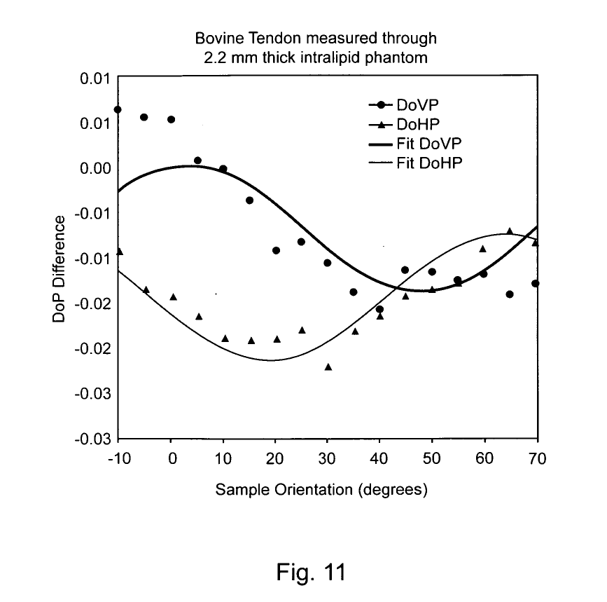

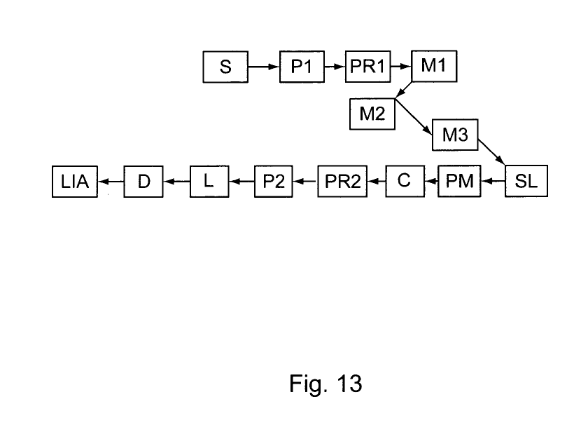

From FIGS. 10 and 11 it can be seen that the measurement of tendon through 0.5 mm thick Teflon or 2 mm thick Intralipid phantoms is approaching the detection limit of the embodiment used. The intervening layers (Teflon or Intralipid) are highly scattering and have the effect of partially depolarizing the light even before it reaches the collagen. In order to detect collagen birefringence through greater thicknesses, the instrument can include automated embodiments such as diagrammed in FIG. 13. The schematic in FIG. 13 shows an embodiment comprising: polarization compensator C (quarter-wave plate), detector D (silicon photodiode with built-in trans-impedance amplifier), lens L (1″ diameter, 75 mm FL), lock-in amplifier LIA (reference to source modulation), mirror M1 (0.5″ diameter round), mirror M2 (0.5″ diameter round), mirror M3 (1″ diameter, D-shaped), polarizer P1 (source polarizer), polarizer P2 (source polarizer), parabolic mirror PM (90 degree off-axis), polarization rotator PR1 (rotating HWP), polarization rotator PR2 (liquid crystal), laser source S (808 nm, 200 mW, 1 kHz, square-wave modulated), sampling lens SL (split lens, 1″ diameter PCX, 15 mm FL, 1 mm black ABS spacer).

In such embodiments, rather than setting the sample orientation with a manual rotation stage, polarization rotator PR1 can be added just after first polarizer P1. Such rotator device can comprise a half-wave plate mounted in a computer-controlled rotation stage. This arrangement allows the sample to remain stationary while the relative polarization of the input beam is varied (e.g., by rotating PR1). Such embodiments are thought to have the advantage of interrogating the same sample volume throughout the measurement, whereas in other embodiments the sample is moved relative to the input beam each time the sample rotation stage is adjusted. Thus, in embodiments comprising such rotators, in addition to the advantage of automating the measurement, the measurement error is also thought to decrease. Also, in the current embodiments, when the laser beam is oriented at angles other than 0 (vertical) and 90 degrees (horizontal), mirrors M1-M3, PM, and the sampling lens cause partial depolarization of the light. Therefore, placing quarter-wave plate C in front of second polarizer PR2 largely compensates for this depolarization effect. In other embodiments, additional compensators can be also used (e.g. a quarter-wave plate between PR1 and M1) in order to further increase instrument sensitivity.

### Software Flow Diagram

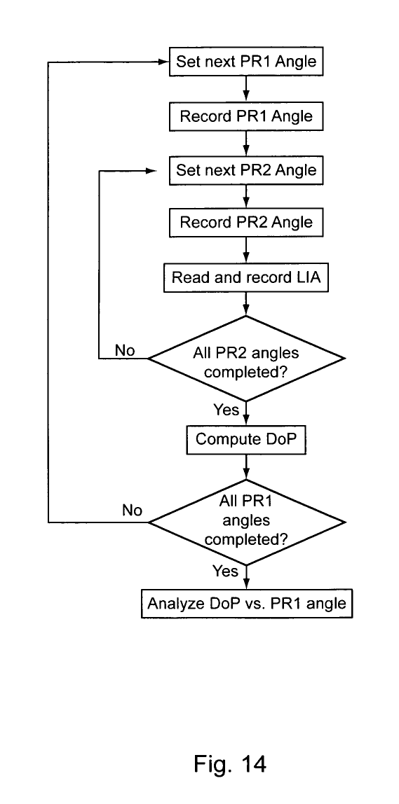

An exemplary flow diagram summarizing and generalizing the steps involved in automated measurement of the orientation of a collagen sample (e.g., as with the embodiment characterized in FIG. 13 using determination of DoLP or DoVP/DoHP) is shown in FIG. 14. In the diagram, the PR1 angle refers to the orientation of the input linear polarization relative to the birefringent axis of the collagen sample, while the PR2 angle refers to the polarization rotation induced by a particular voltage setting of the liquid crystal polarization rotator. It will be appreciated that the flowchart in FIG. 14, and indeed the various embodiments of the methods/devices herein can involve linear or circular polarization. Degree of Polarization (DoP) is a general term used to describe any of the following: Degree of Linear Polarization (DoLP), Degree of Vertical Polarization (DoVP), Degree of Horizontal Polarization (DoHP), or Degree of Circular Polarization (DoCP). In an alternative embodiment the polarization rotation angle rather than the DoP is computed. The computer component in various embodiments of the invention can comprise this and/or similar software instruction sets. In various embodiments, the software/instruction set used for monitoring tissue status can optionally comprise a threshold value (either manually set by a user or pre-set within the software, e.g., at the time of manufacture/programming). Such threshold value can be the maximum amount of tissue change (e.g., collagen denaturation) allowed for a treatment. Thus, when an embodiment of the invention determines that a tissue being monitored has reached the threshold value, the systems of the invention can produce a warning to a user of the system indicating that the threshold has been reached and that treatment should therefore stop. In other embodiments, the systems of the invention can be operably connected with the systems/devices used for the treatment of the tissue and can automatically stop treatment when the threshold has been reached. Other information of thresholds is given below in the description of embodiments comprising determination of amount of polarization rotation. It will be appreciated that such threshold aspects are also optionally applicable to the embodiments herein comprising determination of DoLP, etc.

### Alternative Instrument Configurations

The various instrument configurations described in the embodiments comprising determination of DoLP, polarization angle, etc. are designed to allow rapid measurement of the degree of linear polarization in a tissue (e.g., a tendon) sample. The measurements being collected by the various embodiments include 3 of the 4 Stokes vectors, (see, e.g., Kliger, et al., “Polarized Light in Optics and Spectroscopy”, Academic Press (New York), 1990) whereas a full Stokes measurement would include circular polarization in addition to linear. Since the relative input linear polarization is also being varied in the methods described here, the measurement can further be characterized as a partial determination of the Mueller matrix. See Kliger, supra. Measurement of the full 4×4 Mueller matrix requires characterization of all 4 Stokes vectors while varying the input polarization between at least 4 characteristic states of linear and circular polarization. Thus, in some embodiments of the invention, the methods and devices of the invention can comprise measurement of the full 4×4 Mueller matrix in monitoring tissue birefringence. Such embodiments can comprise, e.g., rapidly rotating quarter wave plates (e.g., in both the excitation and detection arms of the systems) to be used along with Fourier analysis. Other embodiments can comprise photoelastic polarization modulators or liquid crystal retarders instead or rotating quarter wave plates. In some embodiments Fourier analysis can be used to determine the elements of the Mueller matrix.

### Monitoring of Multiple Tissues in Embodiments Comprising Determination of Degree of Depolarization

As is common to both general embodiment classifications herein, the embodiments wherein tissue orientation is determined and monitored through measurement of the extent or degree of depolarization can be used to simultaneously or concurrently monitor two or more tissue structures (e.g., a tendon and an overlying dermal collagen layer). Thus, in such embodiments, the point where DoLP (or DoVP/DoHP) is determined to be at a maximum corresponds to where the polarized light beam is parallel with, e.g., the collagen strands in a tendon. The amount of DoLP, etc. at that point is mainly due to, e.g., dermal, synovial, or mucosal collagen (e.g., from an overlying layer above the tendon). Thus, any change to this maximum reading can also be tracked during treatment to thereby monitor any changes in the overlying structure. In some embodiments wherein multiple tissue layers are monitored at the same time, the DoP can be characterized as a function of orientation angle. Thus, rather than just looking for a minimum or maximum the function would be fit to a model. In addition, in some embodiments it can be helpful to fit the DoP as a function of orientation angle to a model, where the model includes depolarization effects from both layers. Some simple examples of such models include: (1) A planar collagen layer plus a linear collagen layer can be modeled as a combination of an angle independent depolarization (planar layer) plus a sinusoidally varying DoP with sample angle (linear layer). (2) Two stacked linear collagen layers can be modeled as a combination of 2 sinusoidally varying DoP functions that differ in phase (corresponding to a difference in the orientation of the optical axes of the two layers). (3) A partially linear/partially planar collagen structure plus a fully linear collagen structure can be modeled as a small amplitude sinusoid plus a large amplitude sinusoid, again with phase difference indicative of the orientation of the birefringent axes. In more detailed models, the orientation dependence of the DoP resulting from each birefringent structure can be modeled as a distribution of sine functions (or other periodic functions), in order to account for the degree of disorder in the collagen orientation within the structure.

The various embodiments comprising determination of DoP demonstrate that the angular orientation of a tendon sample can be determined with high accuracy (<<5 degrees) by measuring the degree of linear polarization while varying the angle of input polarization relative to the sample orientation. As shown by the Examples, this angular variation has the expected 90 degree periodicity and diminishes as the tendon sample is heated (denatured). Also as demonstrated, measurement of tendon samples with embodiments of the invention through several skin “phantoms” can be done including measurement through: (1) at least 7 mm thick gelatin, (2) at least 0.5 mm thick Teflon, and (3) at least 2 mm thick 2% Intralipid (which has light scattering properties similar to human skin, see Troy, supra). The invention also provides a method for simultaneously fitting DoVP and DoHP curves which provides better reliability than use of DoLP alone in some embodiments. Embodiments of the invention also showed measurement of dermal collagen in skin.

### Orientation of Monitoring Polarization Relative to Collagen Axis

The other general classification of embodiments herein comprises determination of tissue (e.g., collagen in a tendon) orientation by tracking the amount of polarization rotation at a number of angles. In some such embodiments, the invention comprises methods and devices/systems to simultaneously or concurrently monitor multiple tissue structures. Thus, for example, some embodiments can monitor a collagen tendon while also monitoring an overlying dermal collagen layer. In some such embodiments, the monitoring comprises determining (on a multi-layer tissue such as tendons under skin) the linear polarization orientation (of the applied light used) at which birefringence of the reflected light from the targeted structure (i.e., the structure that is targeted for treatment, such as linear collagen in a tendon) is minimized. Minimum birefringence will be observed when the light polarization of the applied light is oriented parallel to the linear strand direction (or at 90 degree increments from such polarization). In such embodiment, the birefringence measured at the polarization orientation that gives minimal birefringence is mainly due to dermal, synovial, or mucosal collagen in the illustration. In other words, the birefringence seen is that arising from the planar overlying layer of collagen rather than from the targeted structure such as a tendon. In such embodiments, in order to achieve greatest sensitivity to the birefringence from light reflected from the structure targeted for treatment (e.g., sub-dermis, tendon, ligament, fascia, aponeurosis, etc.) the polarization of the applied light can be rotated by 45 degrees from the prior determined angle. As will be appreciated, the ability to track changes in birefringence (e.g., arising from structural changes such as denaturation) in more than one structure allows monitoring to ensure that not only is the targeted structure not altered beyond a particular point (and/or that a targeted structure is altered up to a particular point), but also that another particular structure (e.g., an overlying one) is not altered beyond a particular point (or is altered up to a particular point) as well.

In such embodiments, the polarization of a laser relative to the strand axis of a tendon (or other highly oriented collagen material being targeted by treatment such as RF) is first oriented, by searching for the angle of minimum (or maximum) birefringence. Once oriented, the degree of polarization rotation induced by the collagen at 45 degrees vs. 0 degrees is used to contrast the birefringence of the tendon relative to that of more superficial dermal collagen. The search procedure for both locating the optic axis of the collagen and for monitoring its subsequent changes in response to treatment such as RF heating, involves rotation of the angle of a polarizer in front of a detector, while searching for a minimum signal level.

An illustrative embodiment of system components to implement such embodiments is diagrammed in FIG. 1. Although not shown in the figure, the laser light is launched vertically into the sampling lens (SL) and light captured from the sample is launched back into the horizontal plane by the parabolic mirror (PM). In the remainder of the apparatus the laser light is confined to a horizontal plane. It will be appreciated that embodiments can also typically comprise additional components, e.g., computers/processors, rotation drivers, etc., such as those illustrated in FIGS. 16 and 17. The components shown in FIG. 1 include: detector D (silicon photodiode with built-in trans-impedance amplifier); lens L (1″ diam, 75 mm FL); lock-in amplifier LIA (reference to source modulation); mirror M1 (0.5″ diam. round); mirror M2 (0.5″ diam. round); mirror M3 (1″ diam., D-shaped); polarizer P1 (source polarizer); polarizer P2 (detection polarizer); parabolic mirror PM (90 degree off-axis); polarization rotator PR (liquid crystal); laser source S (808 nm, 200 mW, 1 kHz, square-wave modulated); and sampling lens SL (split lens, 1″ diam. PCX, 15 mm FL, 1 mm black ABS spacer).

A sample consisting of a half-wave plate with a mirror on top, was used to test the performance of the instrument. As expected, at 0 degree orientation, the sample had no effect on the laser beam polarization, whereas at 45 degree orientation, the sample rotated the laser beam polarization by approximately 90 degrees. In addition to these expected findings, it was also unexpectedly found that the degree of linear polarization was being substantially reduced at the 45 degree orientation. Further experiments traced the source of this unexpected depolarization to 2 factors: (1) mirror reflections at angles other than 90 degrees were causing mixing of polarization states, and (2) optical imperfections in the sampling lens were causing some polarization scrambling. Use of quarter and half-wave compensator optics can thus be used to correct such effects.

When changing the sample to bovine tendon (frozen after harvesting; then thawed in phosphate buffered saline before measurement) a high degree of depolarization was observed at all orientation angles with the system arrangement in FIG. 1. The high degree of depolarization made it difficult to find the minimum polarization angle (P2) needed to determine the extent of sample-induced polarization rotation. Using very thin (˜1 mm) slices of collagen in conjunction with a mirror (placed on top of the sample to reflect back the laser light), some rotation effects were observed (not shown). The effects had less than desirable reproducibility and accuracy. With thicker collagen samples, depolarization made it difficult to reliably identify the minimum polarization angle, particularly at the 45 degree orientation using the current embodiment. Many of such difficulties can be addressed through use of the embodiments outlined above using determination of DoLP, DoVP/DoHP, or polarization angle using Stokes vector analysis.

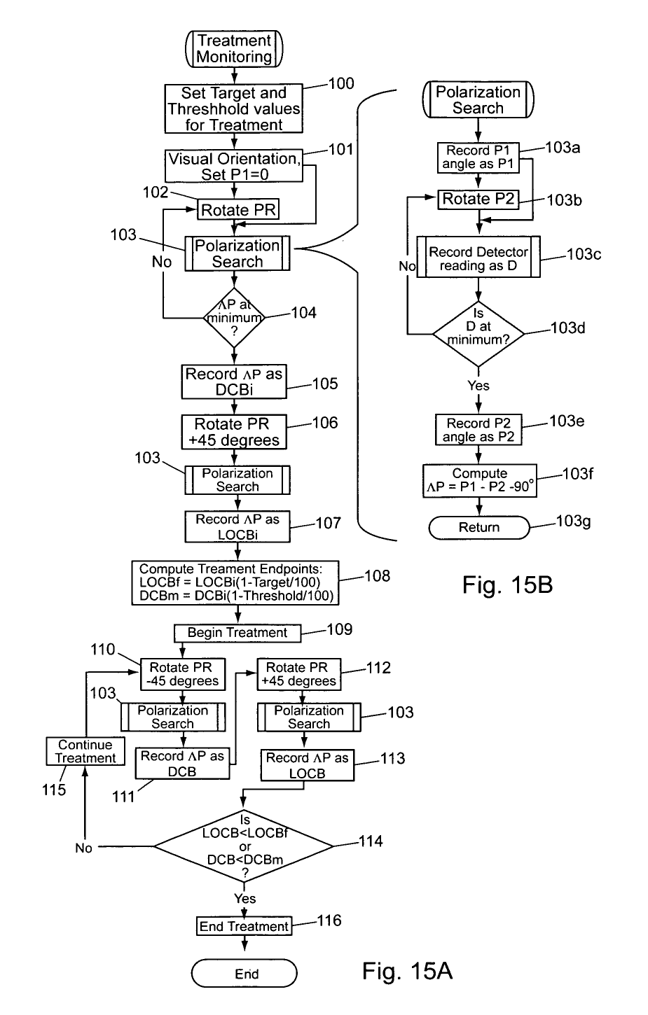

The flowcharts in FIG. 15 illustrate the major actions involved in the current embodiments using determination of amount of polarization rotation. As can be seen in FIG. 15, treatment monitoring by various embodiments of the current invention can comprise a number of steps. As seen in FIG. 15A, prior to beginning treatment of a tissue (or alternatively even once treatment has begun), both Target and Threshold values can be chosen. See FIG. 15A Box 100. A Target value herein indicates a desired level of treatment of a particular structure or tissue. For example a Target value could be, e.g., 10% collagen denaturation of an area of tendon. A Threshold value herein indicates a maximum level of treatment of a particular structure or tissue. A Threshold value is typically the point of treatment beyond which damage will or could occur. Of course, it will be appreciated that in practical use, Threshold values will typically be set with various safety factors in mind. Thus, for example, a Threshold value could be, e.g., 30% collagen denaturation of an area of a dermal collagen. Such value can indicate the point beyond which damage or undesirable results could occur plus an additional safety zone.

The light reflected from the structures within the tissue can be collected via a second polarization preserving optical fiber (e.g., fiber 1609 in FIG. 16). The spacing between the first and second optical fibers can be chosen to be, e.g., approximately equal to the midpoint of the depth of the structure (e.g., tendon) that is targeted for treatment. Thus, for example, if the target to be treated is a tendon lying 2 mm below the skin surface and having a 2 mm thickness, the spacing between the first and second optical fiber can optionally be approximately 3 mm. It will be appreciated that different embodiments having different configurations can comprise different placements/arrangements. The second optical fiber directs the reflected light through an optional second light polarizer (e.g., 1607 in FIG. 16) and into a light detector (e.g., detector 1605 in FIG. 16).

Once Target and Threshold values are determined, the polarization of the light can optionally be oriented to be parallel to the linear organization of a target structure (e.g., oriented with the direction of linear organization in a tendon to undergo treatment). See Box 101. Of course, such “matching up” is optional and the starting measurements can begin at any polarization orientation. In either embodiment, the resulting polarization measurement (i.e., the measurement of the polarization of light reflected back from the tissue/structure being treated) is measured once it reaches the detector.

After the optional visual orientation, a polarization search (Box 103 and substeps) is performed. In the polarization search, the angle of polarizer 1601, see FIG. 16, is recorded and the resulting light intensity is measured at the detector. The second polarizer 1607 is then rotated until the minimum detected intensity is reached. In one example implementation, polarizer 1607 is first stepped across an approximately 50 degree range in approximately 5 degree increments. Next, centering on the polarization where minimum intensity was observed, polarizer 1607 is stepped across a 10 degree range in 1 degree increments. This process is continued, with both the range and step size diminishing by a factor of approximately 5 at each stage, until the minimum detected intensity has been located with approximately millidegree sensitivity. As an alternative to the above-described “simplex” type of search process, a “gradient” search may be instead employed. Similarly diminishing step sizes are employed in the gradient search, but the direction of each new step is informed by prior steps as to the direction of the expected minimum. This later method has the advantage of speed, whereas the former method has the advantage of being less susceptible to the detection of local minima. Once the minimum detected intensity is observed, the angle of polarizer 1607 is recorded and subtracted from the original angle of polarizer 1601 and 90 degrees subtracted from such difference. The polarization rotating device 1602 (see FIG. 16) is then rotated incrementally and the substeps of Box 103 repeated. See Box 102. As with the “Polarization Search” described above, in an example implementation, initially large (approximately 5 degree) steps are diminished to 1 degree steps following either a simplex or gradient search. In this case however, polarization rotation steps smaller than about 1 degree will be unnecessary for most applications. Once ΔP is determined to be at a minimum for that location, the angle is recorded as DCBᵢ, or the initial or baseline birefringence, e.g., of the overlaying collagen layer. The setting of the polarization-rotating device at such reading is set as position “0.” Next, the polarization rotating device is adjusted so that the polarization is rotated precisely 45 degrees (i.e., position “45”). See Box 106. The substeps of Box 103 are then repeated for the new position of the polarization rotating device. The minimum ΔP for “position 45,” i.e., the initial linearly-oriented collagen birefringence, or LOCBᵢ, is thus determined:

Once, or even before, the LOCBᵢ and DCBᵢ have been determined, Target values are set for the percent change desired for the linearly oriented collagen. As stated, the values may be previously set values, or may be values selected by the user through a user interface. From the desired percent change, the “final linearly-oriented collagen birefringence,” or LOCB_{f}, is calculated by:

LOCB_{f}=LOCBᵢ(1−Target/100) Equation 9

Next a Threshold value for the desired maximum allowable percent change in the DCBᵢ is determined. Again, this can be a stored value, or can be selected through a user interface. From the maximum percent change the “minimum dermal collagen birefringence,” or DCBₘ, is calculated by;

DCBₘ=DCBᵢ(1−Threshold/100) Equation 10

After the above values are determined, treatment of the tissue/biological structure(s) begins. See Box 109. As explained throughout, the particular treatment that is monitored by the current invention should not be taken as limiting. In particular embodiments, the treatment comprises RF energy with surface cooling.

Simultaneous with the treatment application, steps 110 through 115 are performed in particular embodiments. Thus repeated birefringence measurements with the polarization-rotating device alternately set the “0” and “45” positions are done. The birefringence measurements are optionally performed at a rate that is high (e.g., in a millisecond time scale) compared to the effect of the treatment. Alternatively, the treatment application and birefringence measurements can be performed sequentially, with each treatment step being small (e.g., less than 1/10^{th} of the target percent change) compared to the overall desired treatment level. In various embodiments, the monitoring footprint is smaller than the treatment footprint, while in other embodiments, the monitoring footprint is optionally the same size as or even bigger than the area being treated. It will be appreciated that in some embodiments, the monitoring can be done at numerous set positions simultaneously or sequentially during the treatment to monitor a broader area of the treatment footprint.

As indicated in steps 114 through 116, treatment is stopped when either the birefringence measurement with the polarization-rotating device set to the “45” position falls below the target setting, LOCB_{f} or when the birefringence measurement with the polarization-rotating device set to the “0” position falls below the minimum setting, DCBₘ. Alternately, a secondary threshold can be used to signal a stop in treatment. For example, the secondary threshold may be based on the length of time of treatment or the total energy deposited.

In addition, a feedback loop is optionally used to independently adjust the amount of heating (energy) or cooling delivered to the tissue being treated to maximize the desired changes in LOCB while minimizing the undesirable effects on DCB.

### Other Methods and Algorithms for Monitoring Birefringence

Besides the measurements and computed functions described elsewhere in this document for assessing tissue birefringence (e.g. DoLP, DoVP, DoHP, Δθ), numerous alternative methods and functions may also be effective. In order to further describe these measurements and methods, three angles are considered: (1) δ, the angle between the linear polarization axes of the excitation and detected light, (2) φ, the detection polarization angle (relative to vertical polarization), and (3) γ, the angle of rotation of the sample within the sample holder (positioned just above the sample lens). In what follows, the detected light intensity as a function of angles is denoted as I_{γ} (φ, γ). In certain embodiments described above, measurements made at different detection polarization angles, φ, were used to compute a function such as the degree of polarization (DoP), and then the dependence of the DoP on the sample angle, γ, was used as an indicator of sample birefringence. More generally, however, a DoP function can be determined by selecting one variable out of any of δ, φ, or γ and making at least 2 measurements of detected intensity as a function of this variable. Then, by choosing a 2^{nd} variable out of the same set (δ, φ, and γ) and measuring the DoP function at 2 or more different values, the birefringence of the sample may be assessed.

In one embodiment, the angle between the linear polarization axes of the excitation and detected light, δ, is varied between two angles, 90 degrees apart. The DoP function is computed from these two measurements according to:

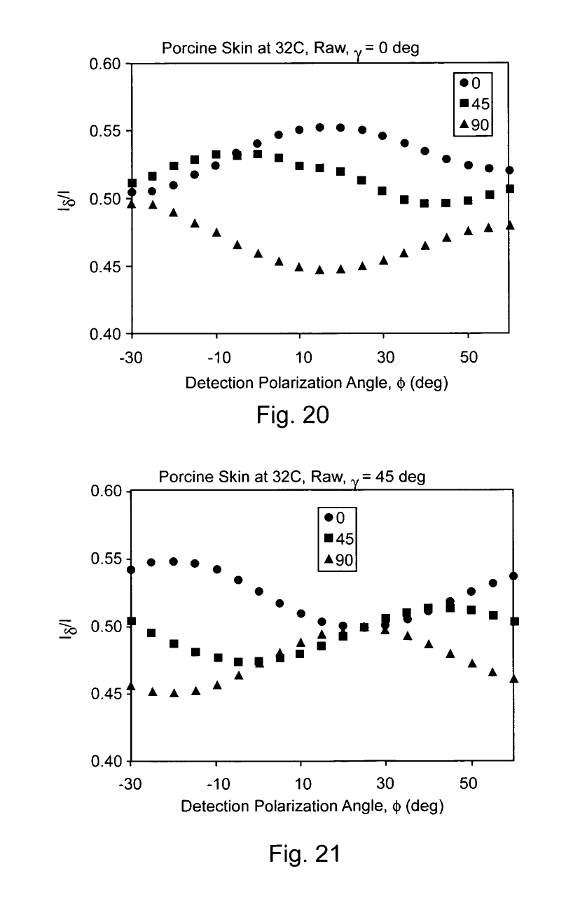

DoP(φ,γ)=Iₓ(φ,γ)/I(φ,γ) Equation 11

where I(φ,γ)=Iₓ(φ,γ)+Iₓ₊₉₀(φ,γ). The detection polarization angle, φ, is then varied across a range of angles with DoP being re-measured at each new angle. A measurement of porcine skin is shown in FIG. 20 (Example 5), with φ varied between −30 and 60 degrees in 5 degree steps. Once the angular dependence, DoP(φ), has been determined, then repeated measurements of DoP at 2 or more angles, φ, can be used to monitor for changes in the birefringence of the sample. For example, in FIG. 20, by repeatedly measuring I₀/I with φ at −30 and 15 degrees, the difference between the DoP at the two angles, is indicative of the degree of birefringence, DoB, of the sample:

DoB(φ,γ)=DoP(φ₁,γ₁)−DoP(φ₂,γ₂) Equation 12

Rather than varying the detection angle, φ, to determine the degree of birefringence, the sample orientation angle, γ, could instead be varied. A comparison of FIGS. 20 and 21, measured at γ angles 45 degrees apart, illustrates this point. The value of I₀/I determined while holding φ fixed at 20 degrees and varying γ between 0 and 45 degrees, produces an equivalent result to fixing γ while varying φ. Also, as can be seen in FIGS. 20 and 21, instead of choosing I₀/I as an indicator of the DoP, I₉₀/I or I₄₅/I could be equivalently used. The DoP function can thus be defined as any combination of at least two measurements of I_{δ} (φ,γ). In this manner, DoP is used as a generalized term which also encompasses the terms DoLP, DoVP, DoHP, DoCP as defined elsewhere in this document. In many cases, the measurements of I_{δ} (φ,γ) used to determine the DoP will include 2 measurements at polarization angles (e.g. δ, φ or γ) that are 90 degrees apart. Such a measurement allows for an angle-independent assessment of intensity:

I=Iₓ+Iₓ₊₉₀ Equation 13

In many cases, it will be useful to use the angle-independent intensity, I, to normalize the angular-dependent intensities, I_{δ} (φ,γ) in computing a DoP function, so that the function is independent of the intensity of the light source used to perform the measurement.

Alternate DoP functions include ratios of the intensities measured at 2 angles:

DoP=I_{δ1}(φ₁,γ₁)/I_{δ2}(φ₂,γ₂) Equation 14

where at least one of the angles δ, φ, and γ is different for the two measurements. In one embodiment a ratio of the intensities measured with the angle δ at 2 values 90 degrees apart is used as the DoP function:

DoP=I_{δ}(φ₁,γ₁)/I_{δ+90}(φ₁,γ₁) Equation 15

In a similar manner the DoB function is defined as any combination of at least two measurements of DoP. In many cases the measurements of DoP will include 2 measurements at angles (e.g. δ, φ or γ) that are 45 degrees apart. This is because for a uniaxial birefringent material such as collagen, the maximum and minimum birefringence are expected to occur 45 degrees apart. In one alternate embodiment, the DoB function is computed from a ratio of DoP measurements determined at 2 or more angles:

DoB=DoP(1)/DoP(2) Equation 16

### Instrument Calibration

Besides the sample being measured, components of the instrument may also cause depolarization of the light being measured. Half-wave and quarter-wave plates may be used to compensate for this depolarization. For example, it is expected that when a mirror is used as the “sample” the DoLP should have a unity value at all orientation angles. Referring to “FIG. 2”, for example, with a mirror on the sampling lens, SL, the compensators, C1 and C2, can be rotated about the axis of the transmitted light to maximize the measured DoLP. This process is then repeated at all excitation polarization angles to be measured.

Alternatively, or in addition to the use of compensator optics, numerical methods for correcting the measured birefringence may also be applied. In one embodiment a multiplicative correction is determined using a mirror in place of the sample, and in subsequent measurements the correction is applied to other sample types. Example algorithms for applying multiplicative correction to the measured intensity, I, are:

$$\begin{matrix}{( \frac{{I}_{0}^{S}}{{I}^{S}} )}^{′} = ( \frac{{I}_{0}^{s}}{{I}^{S}} ) ( \frac{{I}^{M}}{{I}_{0}^{M}} ) & Equation 17 a \\ {( \frac{{I}_{90}^{S}}{{I}^{S}} )}^{′} = 1 - {( \frac{{I}_{0}^{s}}{{I}^{S}} )}^{′} & Equation 17 b \\ {( \frac{{I}_{45}^{S}}{{I}^{S}} )}^{′} = ( \frac{{I}_{45}^{s}}{{I}^{S}} ) ( \frac{{I}^{M}}{2 {I}_{45}^{M}} ) & Equation 17 c\end{matrix}$$

where the superscripts S and M refers to the sample and mirror, respectively and the subscript refers to the polarization angle (any of δ, φ, or γ). In the absence of a subscript, the symbol l refers to the angle-independent intensity, which may be computed according to:

l^{S}l₀^{S}+l₉₀^{S} Equation 18 a

l^{M}l₀^{M}+l₉₀^{M} Equation 18b

In other embodiments, the numerical correction algorithm that is suitable may be additive rather than multiplicative:

$$\begin{matrix}{( \frac{{I}_{0}^{S}}{{I}^{S}} )}^{′} = ( \frac{{I}_{0}^{s}}{{I}^{S}} ) + 1 - ( \frac{{I}_{0}^{M}}{{I}^{M}} ) & Equation 19 a \\ {( \frac{{I}_{90}^{S}}{{I}^{S}} )}^{′} = 1 - {( \frac{{I}_{0}^{s}}{{I}^{S}} )}^{′} & Equation 19 b \\ {( \frac{{I}_{45}^{S}}{{I}^{S}} )}^{′} = ( \frac{{I}_{45}^{s}}{{I}^{S}} ) + \frac{1}{2} - ( \frac{{I}_{45}^{M}}{{I}^{M}} ) & Equation 19 c\end{matrix}$$

In further embodiments, a correction algorithm with both additive and multiplicative terms is suitable:

$$\begin{matrix}{( \frac{{I}_{0}^{S}}{{I}^{S}} )}^{′} = a ( \frac{{I}_{0}^{s} + c}{{I}^{S}} ) + ( 1 - a ) ( \frac{{I}_{90}^{s} - c}{{I}^{S}} ) & Equation 20 a \\ {( \frac{{I}_{45}^{S}}{{I}^{S}} )}^{′} = b ( \frac{{I}_{45}^{s} + d}{{I}^{S}} ) + ( 1 - b ) ( \frac{{I}_{- 45}^{s} - d}{{I}^{S}} ) & Equation 20 b\end{matrix}$$

Correction factors a and d may be determined by using a mirror in place of the sample and setting the relative angle between the excitation and detection polarization axes, δ, to zero. Correction factors b and c may also be determined using a mirror in place of the sample, but with the angle δ set to 45 degrees. The correction factors may then be computed from the mirror measurements according to:

$$\begin{matrix}a = \frac{{I}_{0}^{M} ( 0 ) + c}{{I}_{0}^{M} ( 0 ) - {I}_{90}^{M} ( 0 ) + 2 c} & Equation 21 a \\ b = \frac{{I}_{45}^{M} ( 45 ) + d}{2 {I}_{45}^{M} ( 45 ) - {I}^{M} ( 45 ) + 2 d} & Equation 21 b \\ c = \frac{{I}_{90}^{M} ( 45 ) - {I}_{0}^{M} ( 45 )}{2} & Equation 21 c \\ d = \frac{{I}_{- 45}^{M} ( 0 ) - {I}_{45}^{M} ( 0 )}{2} = \frac{{I}^{M} ( 0 ) - 2 {I}_{45}^{M} ( 0 )}{2} & Equation 21 d\end{matrix}$$

where parentheses are used to indicate the angle δ.

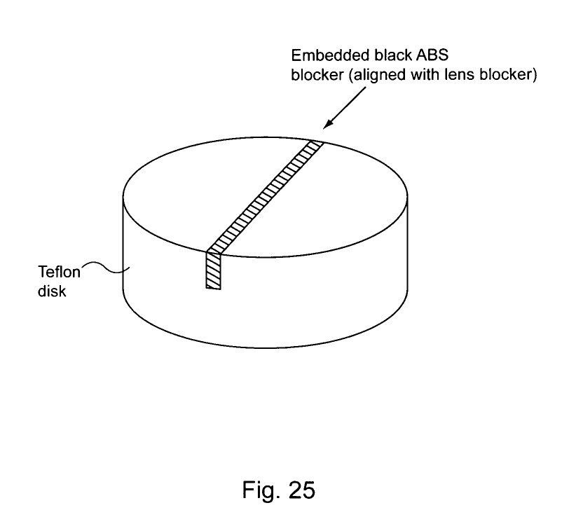

The above-described algorithms employ a mirror in place of the sample with the expectation that the mirror will reflect the excitation light without substantially depolarizing it. In an alternative embodiment a material that is expected to be fully depolarizing is instead employed as the calibration standard. One standard suitable for this purpose is a Teflon block. The Teflon block may optionally include a material embedded in the block that forces the excitation photons to travel through a long path before reaching the detection optics, thereby ensuring substantial depolarization of the detected light. Suitable materials are substantially non-transmissive of the excitation light. An example of such a material is a strip of black ABS plastic, embedded to a depth of several millimeters in the Teflon block. FIG. 25 depicts one embodiment of this calibration standard. When employed in conjunction with a split sampling lens (FIG. 18A), the embedded ABS blocker is arranged so that it is aligned with the blocker embedded in the lens. In this manner, the excitation photons must travel through the Teflon and around the ABS in order to reach the detection optics. The correction algorithms described above in conjunction with mirror measurements are also applicable to measurements with a depolarizing reference material, except, rather than correcting the measured intensities to be fully polarized, they will be corrected to be fully depolarized.

In other embodiments of the invention, reference materials providing known amounts of depolarization are employed, such as quarter and half wave plates. In other embodiments, combinations of standards are measured, where to standards provide multiple levels of known depolarization.

In yet further embodiments of the invention, self-correcting algorithms are applied, that allow multiple measurements made on the same sample to be combined to compensate for instrument imperfections. One such algorithm is described in Example 5. In this example, measurements of I_{δ} on porcine skin as a function of φ are collected at two sample orientations, γ=0° (FIG. 20) and γ=45° (FIG. 21). Instrument artifacts are not expected to be affected by the sample orientation. Therefore the artifacts may be compensated by combining two measurements at different sample orientations, such as by:

$${R}_{δ} ( φ ) = \frac{{I}_{δ} ( φ , {γ}_{45} ) / I ( φ , {γ}_{45} )}{2 {I}_{δ} ( φ , {γ}_{0} ) / I ( φ , {γ}_{0} )} \qquad Equation 22$$

The resulting function, R_{δ} (φ), shown in FIG. 23, is alternate Degree of Polarization (DoP) function to those described elsewhere in this document.

### System Overview

The description of various embodiments of the systems/devices of the invention and their uses herein presents the basic components of the invention in a number of exemplary monitoring arrangements. In various such illustrations, the embodiment is described as arranged to monitor (e.g., as for tracking progress of a treatment) a tissue structure such as collagen in a tendon. In other illustrations, the embodiment is described as arranged to monitor a tissue beneath another tissue (e.g., a tendon beneath a dermal skin layer or a dermal collagen layer beneath the epidermis). In yet other illustrations, the embodiment is described as arranged to monitor both an underlying structure (e.g., a tendon, etc.) and an overlying layer structure (e.g., a dermal layer of collagen) as for monitoring a treatment that could possibly affect both layers (e.g., a transdermal thermotherapy application, e.g., via RF treatment). Of course, it will be appreciated that the various component arrangements in the embodiments should not necessarily be taken as limiting. Also, as further explained herein, the monitoring done by/through use of the embodiments herein can be to monitor treatment to an overlying structural layer or to an underlying structural layer, or to more than one structural layer. The invention can also be used to monitor treatment other than thermotherapy, etc. Finally, while the various embodiments present several devices/components and arrangements, it will be appreciated that not all such features or arrangements are necessarily present in all embodiments unless specifically stated.

FIG. 16 shows a schematic that outlines the basic optical components that are found in a number of exemplary embodiments of the invention. As can be seen in FIG. 16, light source 1600 (e.g., a miniature laser such as a vertical cavity surface emitting laser) emits light which travels through excitation polarizer 1601 and then through polarization rotator 1602 and focusing lens 1603. The light then traverses optional polarization preserving fiber 1604 and enters into a tissue layer. As will be appreciated, depending upon the tissue type, the structures present in the tissue, the strength of the light, etc., the light can penetrate to various depths within the tissue. Once within the tissue, the light is reflected from various structures, exits back out of the tissue and is captured by optional polarization preserving fiber 1609. The light further passes through collimating lens 1608, detection polarizer 1607, detection lens 1606 and into detector 1605.

An overview of several basic electrical components present in various embodiments herein is shown in FIG. 17. In FIG. 17, computer 1703 provides digital control signals for polarizer controllers 1701 (for the excitation polarizer) and 1704 (for the detection polarizer) and polarization rotator controller 1702 which modulates the polarization between two linear polarization states. A current source (e.g., source 1700) can be used to power light source 1600. The current source is optionally a direct current power source. The polarization rotator controller can also provide a modulation signal used for lock-in amplification (LIA) of signals detected by detector 1605. The locked in amplification provides a digital signal that is read in by the computer and reported back to the user. Use of and/or control of the various components as shown in FIGS. 16 and 17 can be guided by a computer software algorithm such as that diagrammed in FIG. 14 or 15.

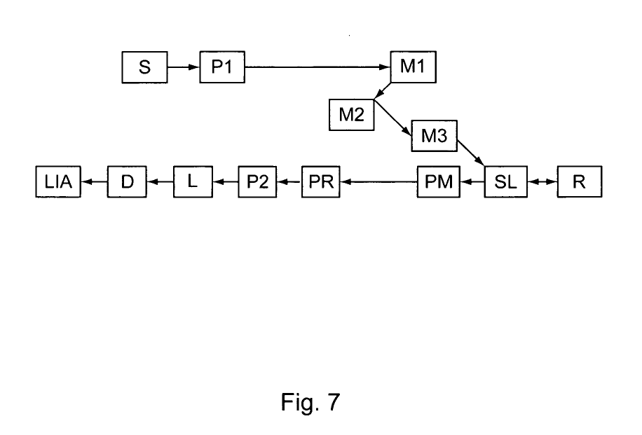

As stated previously, not all embodiments will necessarily comprise all elements/components listed herein or described in FIGS. 16 and 17. For example, a lock-in amplifier, such as element 1706, is optional in some embodiments, although such element can be helpful for distinguishing the signal of interest from other competing noise sources (e.g., room lights, line noise, etc.). Also, measurements of the signal produced by an optional trans-impedance amplifier (e.g., amplifier 1705) can optionally be synchronized with the modulated reference signal. In some embodiments, the light source is modulated and this modulation signal is also used as reference for signal detection. Correspondingly, it will be appreciated that other embodiments comprise additional components than those shown in FIGS. 16 and 17. For example, various embodiments can comprise polarization compensators, split sampling lenses, etc. See, e.g., FIGS. 2, 7, etc.

It will also be appreciated that the digital and analog signals labeled in the Figures should not be taken to indicate strict requirements and that any of the various connections is optionally analog or digital. Digital signals are convenient for long-distance corruption-free transmission of signals, while analog signals are more often used over shorter distance to accomplish the direct interface with mechanical or optical components.

The use of an excitation polarizer, e.g., polarizer 1601, and its controller, e.g., controller 1701, are also optional in some embodiments. In some embodiments, if the light source is already inherently polarized (as is typical for lasers), then these components can be optional. Furthermore, in some embodiments, even if an excitation polarizer is included, its controller is optional. Similarly, in some embodiments a controller for the detection polarizer is optional.

The location of the polarization rotator (e.g. 1602 in FIG. 16) is changed from the excitation to the detection arm of the instrument in some embodiments. For example, in one embodiment the polarization rotator is moved between collimating lens 1608 and detection polarizer 1607. In other embodiments polarization rotators are used in both the excitation and detection arms of the instrument. In some embodiments, the use of a polarization rotator removes the need for control of the excitation or detection polarizers, so one or both of the polarizer controllers is removed.

Although optical fibers (e.g., 1604 and 1609 in FIG. 16) provide a convenient means of transporting light into a device, various “free-space” embodiments of the systems are also included in the invention. Such optional free space systems comprise the benefit of avoiding the inevitable losses associated with coupling light into optical fibers. The lenses required for coupling light into (e.g., lens 1603) and collimating light out of (e.g., lens 1608) the optical fibers are also optional in the free-space embodiments of the device.

In the various embodiments, a lens (e.g., detection lens 1606) to couple light into the detector (e.g., detector 1605) can also be optional. In some embodiments, if the active area of the detector were large enough, the need for such a lens would be removed. However, because there is generally a trade-off between the size of the active area of a detector and its dark noise, the focusing of light onto a detector with small active area is frequently desirable to achieve highest signal to noise ratios.

Additional components and arrangements of components in the systems of the invention are described throughout. For example, FIGS. 1, 2, 3, 7, 13, and 19 present example component arrangements of various embodiments which are described herein.

Light Sources

In typical embodiments herein, the monitoring of tissue (e.g., as in tissue treatment through change in particular biological structures) is accomplished by light excitation and detector or camera observation. It will be appreciated that the invention is not necessarily limited by particular type or specific example of illumination used. Thus, in various embodiments, the light source can comprise, e.g., a laser, an edge-emitting laser diode (e.g., as opposed to a vertical cavity emitting laser diode, VCSEL), a resonant cavity LED, a gas laser (e.g. HeNe), a Nd-YAG laser (e.g., 1064, 532, or 355 nm), an YLF laser (e.g., 1053), a non-laser excitation source such as an LED, halogen or xenon arc lamp, etc.

In the various embodiments, the light source utilized can comprise a monochromatic light of a wavelength that allows for deep penetration into tissue (for example a light having a wavelength of from about 800 to about 1100 nm). Those of skill in the art will be familiar with numerous light emission devices (e.g., vertical cavity surface emitting lasers, VCSEL, having a wavelength of about 850 nm) that can be used in various embodiments of the invention. Depending upon, e.g., the type of tissue to be monitored, whether the monitoring is done topically, endoscopically, or arthroscopically, etc., the intensity/power of the light can be correspondingly chosen or adjusted.

In particular embodiments, the wavelength of light emitted by the light source falls within the range of 700 to 1100 nm. This wavelength region is bracketed on the short wavelength side by regions of strong hemoglobin absorption and on the long wavelength side by increasingly strong water absorption bands. The relatively low tissue absorption observed within the 700 to 1100 nm range allows light to penetrate below the skin layer to probe underlying structures in particular embodiments. Photon penetration depth into a tissue can depend on the scattering and absorption coefficients at the laser wavelength. The optical properties of tissue have been well characterized (see, e.g. “Optical-Thermal Response of Laser Irradiated Tissues,” ed. A. J. Welch, M. van Gemert, Springer, 1995) and many theoretical models have been developed to estimate tissue penetration (see, e.g. “Photon Migration in Tissues,” ed. Britton Chance, Springer, 1989). Furthermore, the penetration depth of detected photons can also depend on the source-detector spacing as discussed further herein.

In some embodiments of the invention, regions of strong absorption by collagen are specifically targeted. Thus, collagen absorption bands can be selectively targeted through larger birefringence effects and use of dichroism effects. Selective absorption by collagen can sometimes be difficult to achieve because in the UV region, other proteins as well as water will contribute to any measured tissue absorbance (or reflectance). Nonetheless, the enhancement of the birefringent effect observed in the UV justifies the choice of this wavelength region in particular embodiments. This is particularly true in applications where superficial collagen structures, such as the dermis, or connective tissue lying just below the skin surface (e.g. elbow) are being targeted. In such cases, the strong absorption of the excitation light can actually serve as an advantage, preventing interference from deeper-penetrating photons which will have had greater opportunity to depolarize through scattering.

Use of UV dichroism in some embodiments of the current invention can require the use of an excitation source(s) with access to a range of wavelengths. Suitable sources can include: (1) a plurality of discrete wavelength sources, (2) tunable sources, and (3) broadband sources whose wavelength region is selected or tuned by a secondary mechanism, such as an optical filter. In some embodiments, the light source can consist of a pair of laser diodes with emission wavelengths at 340 and 400 nm. Collagen absorption at 340 nm is high compared to absorption at 400 nm. See, e.g., Georgakoudi, et al., “NAD(P)H and Collagen as in Vivo Quantitative Fluorescent Biomarkers of Epithelial Precancerous Changes” Cancer Research, 62: 682-687, 2002. The difference between the birefringence observed at the two wavelengths can thus be used as the birefringence signal of interest. Otherwise, the implementation of the invention can be similar to that described for other embodiments herein.

As an alternative to the use of linearly polarized light, circularly polarized light can also be employed in the embodiments herein. Although in particular applications, the use of circularly polarized light can make it more difficult to distinguish between the birefringence due to collagen in skin and that in connective tissue, the triple helical nature of collagen is particularly suited to interact with circularly polarized light and, thus, the increased ease of making the measurement makes its usage beneficial in some embodiments. In such embodiments, no alignment of the polarization relative to the sample is typically necessary, and the measured signal is the differential absorption of the “left” and “right” circularly polarized light.

In other embodiments of the invention, a near infrared protein vibrational absorption band is targeted. For example, by the choice of a light source at 1680 nm, collagen amino acid C-H vibrational absorption can be targeted. Birefringence may thereby be enhanced. Although lipid and water absorption can possibly also contribute to the light absorbance in this spectral region, such contributions can be accounted for in the monitoring processes.

Lenses

In the various embodiments herein, a number of different lenses and lens types are optionally used. For example, split sampling lenses, which are described in more detail above, are used in some embodiments herein. See, e.g., FIG. 7, etc. Furthermore, as also illustrated in FIG. 7, various embodiments herein can comprise lenses that are not split sampling lenses. See, e.g., lens L in FIG. 7. Those of skill in the art will be exceedingly familiar with selection and orientation of lenses suitable for use with the various light sources used in the embodiments herein.

Polarizers

In various embodiments herein, the light used to monitor the tissue can be generally substantially linearly polarized from an emitting device (e.g., a laser). However, particular embodiments can also optionally include a polarizer (e.g., polarizer 1601 in FIG. 16) to increase the polarization extinction ratio by rotating the polarizer for maximum transmission of the light source. As stated, in various embodiments, the polarized light is transmitted from the light source, through an optional polarizer and into a polarization rotating device (e.g., rotating device 1602 in FIG. 16) which allows the user to controllably orient the polarization direction of the light. In typical embodiments, the angle of the resultant polarization of the light transmitted through the polarization rotating device is recorded (e.g., either manually by a user or by the computer component) at the beginning of the monitoring process. The light can then optionally be transmitted through a polarization preserving optical fiber (e.g., optical fiber 1604 in FIG. 16) and onto a tissue site to be monitored (e.g., a tissue undergoing (or that is to undergo) treatment such as thermotherapy). Of course, in other embodiments, the light can be transmitted through “free space” rather than through an optical fiber. Optionally, a user of the invention can orient the optical apparatus so that the polarization is approximately parallel to the long axis of the collagen structure to be treated. Such optional step, whether done manually or whether automated, can aid in reducing the time required for the polarization search steps in particular embodiments.

While the rotation of the polarization can be accomplished by a number of ways, certain embodiments rotate the polarizers and/or polarization rotator either manually or mechanically. Thus, in particular embodiments herein, a polarizer and/or a polarization rotator in a suitable housing is rotated by a suitable DC motor or the like, operating at a speed coordinated with the image capture component. One embodiment of the polarization rotator consists of a wave plate (e.g. half wave plate, quarter wave plate) mounted in a rotatable housing. In yet other embodiments, other devices, such as liquid crystals (such as, but not limited to, those manufactured by Meadowlark Optics (Frederick, Colo.)) or electro-optic or acousto-optic devices (such as, but not limited to, those manufactured by Hinds Instruments (Hillsboro, Oreg.)) can be used to rotate the polarization of the light. With such devices, the polarization can be rotated by varying the voltage or acoustic pulses across the device. Again, control of the rotation can be done manually by a user or can be controlled by the computer component (which, in turn, is optionally controlled by input from the user).

Detection Devices

In the various embodiments herein, the reflected light that returns from the tissues/structures being monitored is captured in a detection device. In turn, the detection device typically relays such information to the computer component of the system. The detection device can comprise, e.g., a CCD camera or the like. Those of skill in the art will be familiar with numerous types and examples of detection devices capable of capture of light emission that can optionally be used with the current invention. Recitation of a particular type or example of detection device in various illustrations herein should therefore not be taken as limiting. For example, the detecting device of the system/devices herein can comprise, e.g., a PIN photodiode (e.g., operated in either photovoltaic or photocurrent modes), an avalanche photodiode, a phototransistor, a photomultiplier tube, or a CMOS array. In the 300-1100 nm spectral range silicon is generally the active detector element for photodiodes. Beyond 1100 nm, silicon becomes unresponsive and alternative materials are typically used. Indium Gallium Arsenide (InGaAs) is an optional choice for detection in the 800-2500 nm range. Germanium is alternate active material for detection in the near infrared spectral region. Thus, various detector devices herein, depending upon the embodiment, can comprise one or more of such elements.

Computer

As noted above, the various components of the present system can be coupled to an appropriately programmed processor or computer that functions to instruct the operation of these instruments in accordance with preprogrammed or user input instructions, receive data and information from these instruments, and interpret, manipulate and report this information to the user. As such, the computer is typically appropriately coupled to these instruments/components (e.g., including analog to digital or digital to analog converters as needed).

The computer optionally includes appropriate software for receiving user instructions, either in the form of user input into set parameter fields, e.g., in a GUI, or in the form of preprogrammed instructions, e.g., preprogrammed for a variety of different specific operations. The software then converts these instructions to appropriate language for instructing the correct operation to carry out the desired operation (e.g., of light illumination and birefringence capture, autofocusing, etc.).

The computer also optionally receives the data from one or more sensors/detectors included within the system, and interprets the data, either provides it in a user understood format (e.g., on a display or computer printout), or uses that data to initiate further instructions, in accordance with the programming, e.g., such as in control of illumination, temperatures, rotation of polarizers, and the like.

In embodiments of the present invention, the computer typically includes software for the monitoring and control of light illumination and capture. Additionally the software is optionally used to control movement of the illumination/capture footprints over a tissue surface, e.g., in coordination with the treatment being monitored. The computer also typically provides instructions, e.g., to any heating/cooling component and autofocus system, etc.

Any controller or computer optionally includes a monitor which is often a cathode ray tube (“CRT”) display, a flat panel display (e.g., active matrix liquid crystal display, liquid crystal display), or the like. Data produced from the current systems, e.g., degree of birefringence or change in birefringence of an area, is optionally displayed in electronic form on the monitor. Additionally, the data gathered from the system can be outputted in printed form. The data, whether in printed form or electronic form (e.g., as displayed on a monitor or deposited on tape, CD, or disc), can be in various or multiple formats, e.g., curves, histograms, numeric series, tables, graphs and the like.

Computer circuitry is often placed in a box which includes, e.g., numerous integrated circuit chips, such as a microprocessor, memory, interface circuits. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

It will be appreciated that the computer component in the systems/devices herein does not necessarily refer to a Personal Computer (PC), but can also or instead comprise a microcontroller or microprocessor.

Automation

Although the methods of the invention can be performed manually, in particular embodiments, such steps as placement of the light and detection components are performed in an automated fashion. Thus, the light emission and capture, polarization changes, adjustment of the light penetration and location on the tissue, etc. are all optionally automated. For example, polarization-rotating devices under electrical control are well known in the field of optics. Automation allows the birefringence measurements to be performed rapidly, so that near-continuous feed-back may be provided for a treatment method being monitored. Furthermore, the birefringence measurement can be performed simultaneously and in close proximity with the application of RF energy due to the weak interaction between RF and optical fields.

Housing

Placement and movement of the various components of the devices/systems herein is optionally controlled and secured by, e.g., an armature, scaffolding, or housing in which the components are located. In particular embodiments, the components, or at least part of the components, are handheld. Handheld and other manipulable components can be used to move over an area to be monitored (e.g., a subject's skin surface) or within an area to be monitored (e.g., within a subject's body). In some embodiments, one or more component of the system comprises a component that can be endoscopically or arthroscopically inserted into a subject. In some embodiments, the systems herein can optionally comprise one or more components to help stabilize and/or locate the one or more other components of the system in relation to the tissue area being monitored. Thus, some embodiments can comprise stabilizers, mounted platforms (e.g., for a subject), straps, etc.

Thus, it will be appreciated that the various components herein, e.g., the light emitting components, heating/cooling components, polarizers, etc., are typically arranged on a scaffolding or framework and optionally enclosed within a housing. The particular configuration of such framework and/or housing can optionally vary in different embodiments based upon, e.g., the particular components, their size, etc. In typical embodiments however, the framework keeps the various components secure and in the proper location and orientation while also optionally aiding in the movement of the components when necessary.

Heating and Cooling

In some embodiments, the systems herein comprise a heating/cooling component (and optionally a heating/cooling control component) having heating/cooling capabilities. In some embodiments, the heating/cooling component can optionally regulate the temperature of the other components of the systems/devices, e.g., the light emitter, the computer, etc. For example, the heating/cooling component can optionally regulate the temperature of the CCD camera. Such temperature control elements can also optionally help regulate the surface of the tissues whose treatment is being monitored.

Probe Geometry

As explained above, different embodiments of the invention can optionally comprise different arrangement of components in the systems/devices. Thus, some embodiments of the invention use more than one source-detector separation in the probe geometry to provide a supplemental or independent method of targeting particular collagen-containing structures. In one embodiment, two source-detector separations are employed. For example, the first source-detector separation and be approximately 1-2 mm, thereby causing the measured birefringence to be primarily due to dermal collagen, while the second source-detector separation can be approximately 2-5 mm, so that the measured birefringence reflects contributions from both dermal collagen and underlying targeted connective tissue structures.

### Birefringence and Optical Monitoring

Polarization-sensitive methods of measuring the structure and conformation of molecules are well known by those of skill in the art. For example, circular dichroism can be used as a method of measuring changes in protein structure. This technique involves measuring the differential absorption of left and right circularly polarized light. Chiral (or “optically active”) molecules will respond differently to these two types of polarization, particularly in wavelength regions matching the energy difference between electronic or vibrational states of the molecule. Far-UV (<250 nm) circular dichroism, in which wavelength range amide electronic bonds are absorptive, can be of particular use in probing the secondary structure of proteins, such as distinguishing alpha-helical, beta-sheet, and globular conformations of proteins. Near-UV (>250 nm) circular dichroism is more sensitive to the protein conformation surrounding aromatic amino acids (tryptophan, tyrosine, phenylalanine) and di-sulfide linkages between cysteine residues.

Linear dichroism is useful for the optical characterization of samples containing molecules aligned in a particular direction. Thus, in some embodiments of the current invention, the differential absorption of linearly polarized light (“parallel” and “perpendicular”) is measured. In wavelength regions matching the energy difference between electronic or vibrational states of the molecule, those molecules oriented with a transition dipole parallel to the light polarization will absorb parallel polarized light more strongly than perpendicularly polarized light.

Linear and circular dichroism are two examples of the more general optical properties of birefringence. In birefringent materials, light of different polarizations travels through the material at different speeds. For linearly polarized light, interaction with a birefringent material can result in rotation of the polarization of the light. The interaction of circularly polarized light with birefringent materials results in elliptically polarized light. Birefringence is referred to as dichroism when light at different wavelengths responds differently to the interaction with the birefringent material. Dichroism effects are generally strongest in spectral regions coincident with the energy separating electronic or vibrational molecular transitions, because in these regions the index of refraction is most rapidly changing. As explained throughout, the current invention uses birefringence in particular embodiments. See above.

### Treatment of Tissue by Drastic Temperature Changes

The methods and systems/devices of the invention can be used in a number of different treatment programs for a number of different medical conditions. Thus, it will be appreciated that recitation of particular treatments or the like herein should not necessarily be taken as limiting. Heat and cold treatments have been widely used in the medical field and numerous well-established therapeutic modalities exist that take advantage of such treatments. See, e.g., Hayashi, et al., “The effect of thermal heating on the length and histologic properties of the glenohumeral joint capsule” Am J Sports Med, 25(1):107-12, 1997; Naseef, et al., “The thermal properties of bovine joint capsule. The basic science of laser- and radiofrequency-induced capsular shrinkage” Am J Sports Med, 25(5):670-4, 1997; and Oloff, et al., “Arthroscopic monopolar radiofrequency thermal stabilization for chronic lateral ankle instability: a preliminary report on 10 cases” J Foot Ankle Surg, 39(3):144-53, 2000. Light, ultrasound, radio frequency, shockwaves, magnetism and other forms of energy have been used to modify tissue temperatures in order to achieve benefits such as stimulation of different biological responses, ablation of tissue, or induction of necrosis. For instance, wound healing response stimulation, collagen stimulation, collagen denaturation, collagen contraction, disruption of fat cells for aesthetic purposes, reduction of benign or malign tumors and the like are all well established medical procedures achieved by temperature variation. The current invention aids such procedures, and others, and increases their usefulness and applicability by allowing monitoring (optionally real time monitoring) of the changes brought about by the treatments. Moreover, in typical embodiments, the current invention can be non-invasive (or minimally invasive) in regard to the tissue being treated, thus avoiding more damaging monitoring processes such as biopsies.

As detailed above, the current invention can, in many embodiments, be used to monitor treatment of collagen structures in various tissues. Besides water, collagen is the main component of skin, cartilage, and connective tissue (including, ligaments, tendons, and the like). Trauma, ageing, and other clinical entities can damage collagen's structure through thinning and disorientation of collagen fibers, myxoid degeneration, hyaline degeneration, chondroid metaplasia, calcification, vascular proliferation, and fatty infiltration, etc. See, e.g., Hashimoto, et al., “Pathologic evidence of degeneration as a primary cause of rotator cuff tear” Clin Orthop Relat Res, (415):111-20, 2003.

As is well known in the art, thermotherapy can cause collagen denaturation and contraction by well-known mechanisms of action. See, e.g., Allain, et al., “Isometric tension developed during heating of collagenous tissues. Relationships with collagen cross-linking” Biochim Biophys Acta, 533(1):147-55, 1978; Allain, et al., “Isometric tensions developed during the hydrothermal swelling of rat skin” Connect Tissue Res, 7(3):127-33, 1980; Chen, et al., “Heat-induced changes in the mechanics of a collagenous tissue: isothermal, isotonic shrinkage” J Biomech Eng, 120(3):382-8, 1998; Cooper, et al., “Correlation of thermal properties of some human tissue with water content” Aerosp Med, 42(1):24-7, 1971; Hayashi, et al., “The biologic response to laser thermal modification in an in vivo sheep model” Clin Orthop Relat Res, (373):265-76, 2000; Hayashi, et al., “The effect of nonablative laser energy on joint capsular properties. An in vitro mechanical study using a rabbit model” Am J Sports Med, 23(4):482-7, 1995; Hayashi, et al., “The mechanism of joint capsule thermal modification in an in-vitro sheep model” Clin Orthop Relat Res, (370):236-49, 2000; and Hayashi, et al., “The effect of nonablative laser energy on the ultrastructure of joint capsular collagen” Arthroscopy, 12(4):474-81, 1996.

The use of collagen denaturation/contraction through energy-based treatment modalities can be used in a number of important applications in fields ranging from neurosurgery and orthopedics to ophthalmology, aesthetic medicine, and surgery of the gastrointestinal tract. See, e.g., Avitall, et al., “Physics and engineering of transcatheter cardiac tissue ablation” J Am Coll Cardiol, 22(3):921-32, 1993; Avitall, et al., “The safety and efficacy of multiple consecutive cryo lesions in canine pulmonary veins-left atrial junction” Heart Rhythm, 1(2):203-9, 2004; Avitall, et al., “Linear lesions provide protection from atrial fibrillation induction with rapid atrial pacing” J Cardiovasc Electrophysiol, 13(5):455-62, 2002; Brodkey, et al., “Reversible Heat Lesions With Radiofrequency Current. A Method Of Stereotactic Localization” J Neurosurg, 21:49-53, 1964; Daoud, et al., “Catheter ablation of ventricular tachycardia” Curr Opin Cardiol, 10(1):21-5, 1995; Lesh, “Interventional electrophysiology—state-of-the-art” Am Heart J, 126(3 Pt 1):686-98, 1993; Moraci, et al., “Trigeminal neuralgia treated by percutaneous thermocoagulation. Comparative analysis of percutaneous thermocoagulation and other surgical procedures” Neurochirurgia (Stuttg), 35(2):48-53, 1992; Saxon, et al., “Results of radiofrequency catheter ablation for atrial flutter” Am J Cardiol, 77(11):1014-6, 1996; Seegenschmiedt, et al., “The current role of interstitial thermo-radiotherapy” Strahlenther Onkol, 168(3):119-40, 1992; and Sweet, et al., “Controlled thermocoagulation of trigeminal ganglion and rootlets for differential destruction of pain fibers. 1. Trigeminal neuralgia” J Neurosurg, 40(2):143-56, 1974. Those of skill in the art will be familiar with other applications/procedures utilizing temperature induced denaturation/contraction of collagen. Such applications/procedures are optionally monitored through use of the methods and systems of the current invention.

One common procedure to denature/contract collagen uses temperature generated through radiofrequency energy. Such energy can be applied to an area to be treated, e.g., through direct contact with the targeted structure via a surgical incision. Recently, some technologies have offered the ability of providing therapeutic heat levels through an overlying tissue (e.g., heat/energy applied transcutaneously, transsynovially, transmucosally, transintimaly, etc.) with the goal not only of treating the desired collagen but also preserving the integrity of non-targeted structures (e.g., skin, subcutaneous tissue, and other tissues depending on the targeted structure). See, e.g., Chen, et al., “Heat-induced changes in the mechanics of a collagenous tissue: isothermal, isotonic shrinkage. J Biomech Eng,” 120(3):382-8, 1998; Naseef, et al., “The thermal properties of bovine joint capsule. The basic science of laser- and radiofrequency-induced capsular shrinkage” Am J Sports Med,” 25(5):670-4, 1997; and Oloff, et al., “Arthroscopic monopolar radiofrequency thermal stabilization for chronic lateral ankle instability: a preliminary report on 10 cases” J Foot Ankle Surg, 39(3):144-53, 2000. However, visualization of the treated structure is often impossible and, even when direct visualization is possible, the fact that most collagen changes are not visible to the naked eye is problematic. Various embodiments of the current invention are especially useful in monitoring such procedures. The invention recognizes the baseline condition of a structure while also determining the impact of the provided treatment on the structure and optionally on other underlying or overlying structures. For example, by distinguishing between the collagen in superficial layers of tissue and the collagen in deeper structures, some embodiments of the current invention can aid in defining the clinical endpoint of a treatment based on changes to the structure being treated and/or on changes to nearby structures.

The fundamental unit of collagen consists of tropocollagen polypeptides organized into a triple helix. This triple helical structure is stabilized by intramolecular bonds, principally hydrogen bonds. The triple helices are further organized by intermolecular bonding. See, e.g., Arnoczky, et al., “Thermal modification of connective tissues: basic science considerations and clinical implications” J Am Acad Orthop Surg, 8(5):305-13, 2000. In tissues such as ligaments and tendons, the association of neighboring helices is largely parallel, resulting in unidirectional strands, while in other tissues such as capsular and dermal collagen, the collagen helices are less unidirectional, being instead confined within a plane.

Application of heat to tissue collagen in the range of 60 to 90° C., results in denaturation of the higher order protein structure. The intramolecular hydrogen bonds stabilizing the triple helices are particularly susceptible to disruption, while the intermolecular bonds are generally more heat stable. Therefore, application of heat has the effect of unraveling the triple helical structure of collagen while maintaining overall strand integrity. The result for ligaments, tendons, and other linearly oriented collagen structures is shortening of the collagen along the long axis and thickening of the collagen along the short axis. See, Arnoczky, supra. For collagen oriented within a plane, such as capsular and dermal collagen, contraction of the planar sheet is observed in response to heat, with thickening in the direction perpendicular to the plane.

During thermal treatments of tissue, the extent of collagen denaturation/contraction is affected by the temperature reached and/or the duration of the treatment provided. See Wall, et al., “Thermal modification of collagen.” J Shoulder Elbow Surg, 8(4):339-44, 1999. In addition, due to the variation in collagen composition, particularly the extent of intramolecular cross-linking, the extent of collagen denaturation/contraction can be difficult to predict in advance even if the temperature profile is fully characterized. Thus, various embodiments of the current invention are beneficial to such treatments because they can monitor the presence and extent of any denaturation that occurs during or as a result of treatment and thereby allows medical practitioners to more accurately administer treatment.

Monitoring of Tissue Changes Arising from Chemical/Cosmeceutical Use

Treatment of tissues through epidermal/dermal or percutaneous application of lotions, creams, or other substances is wide spread in the general populace and within the medical community. Many conditions can be treated by the application of different chemicals that are anticipated to induce changes in tissues including the skin, subcutaneous fat, or connective structures. See, e.g., Fang, et al., “Efficacy and irritancy of enhancers on the in-vitro and in-vivo percutaneous absorption of curcumin” J Pharm Pharmacol, 55(5):593-601, 2003, and Schottelius, et al., “An aspirin-triggered lipoxin A4 stable analog displays a unique topical anti-inflammatory profile” J Immunol, 169(12):7063-70, 2002. For instance, the cosmetics industry provides a wide variety of so called “cosmeceuticals” to increase collagen production. See, e.g., Katayama, et al., “A pentapeptide from type I procollagen promotes extracellular matrix production” J. Biol. Chem., 268(14):9941-9944, 1993. However, no objective measurement of effectiveness has previously been available to users. Thus, the current invention can optionally be used to monitor changes, if any, caused by use of cosmeceuticals or the like. For example, using the present invention, a baseline measurement can be taken of the collagen status of a tissue, the cosmeceutical or other putative treatment can then be applied/performed and additional measurements of the collagen status can be performed to detect any change. Again, it will be appreciated that the invention can monitor change before the treatment, during the treatment, and/or after the treatment of the tissue.

### Monitoring of Other Non-Collagen Tissue

The current invention in similar or alternate embodiments can be used to track other optical properties of tissue for similar purposes (i.e., fluorescence, turbidity and the like). By applying same principles and concepts, but different optical properties and therefore equations, the current invention can be used to measure the baseline content of such a targeted tissue, measure its evolution over time (treated or untreated) and measure the impact of different therapeutic interventions.

For that purpose the current invention will take advantage of reflectance and transmittance of light as it is specific to various tissues. For instance fat has distinct properties than dermis (Near-infrared optical properties of ex vivo human skin and subcutaneous tissues measured using the Monte Carlo inversion technique C Rebecca Simpson et al 1998 Phys. Med. Biol. 43 2465-2478).

As a matter of examples and not to be taken as a limiting list, the following tissues could be monitored by the present invention in its preferred or alternate embodiments: urethra, vaginal wall, uterus, skin, fat, fascias, ligaments, tendons, cartilage, capsules, soft palate, turbines, gastrointestinal tract including sphincters, structures of the airway, and nerves among others.

### Tissue Scattering

By relying on a relative change in birefringence as a monitoring guide to therapeutic action, some embodiments of the invention avoid the pitfalls associated with attempts to non-invasively measure the absolute birefringence of collagen. In particular, skin and tissue are known to strongly scatter electromagnetic waves in the UV to IR range. With each scattering event, the light polarization becomes randomized to some extent, with the result that multiply scattered light may become entirely depolarized. The extent of polarization randomization will affect the size of the measured birefringence signal. Therefore, variations of tissue scattering properties observed from person-to-person or even site-to-site on the same person can make it difficult to make an absolute measure of birefringence of collagen through intervening tissue structures. In various embodiments of the present invention, however, each time the probe is applied, an initial determination of birefringence (as by either the DoP or polarization rotation methods described above) can be made, and RF energy (or any other energy source) can be applied to target a percent change in the birefringence. The number of times that the treatment is reapplied to the same tissue thus determines that net change in collagen denaturation. By relying only on a relative change in the birefringence, as induced by a therapeutic procedure, various embodiments of the present invention avoid the potential inaccuracy associated with an absolute measurement.

Use of the Invention with Various Treatments

As will readily be appreciated by those of skill in the art, the current invention can be used as a diagnostic and/or research tool or in conjunction with therapeutic/prophylactic modalities that aim at changing collagen characteristics. In such embodiments the invention can measure collagen's birefringence in various ways at a baseline and can be used for repeated measurements to establish changes in collagen content or characteristics within the studied tissue to determine the impact of a given intervention. As stated above, the treatment that is tracked with the current invention can treat one or more layers of tissue (e.g., collagen). In situations where only one structural layer is treated, such layer can be superficial to a structural layer that is to not be treated or vice versa. The treatments monitored through the current invention can be those that induce wound healing, induce collagen denaturation/renewal, induce collagen deposition, etc. Again, recitation of particular treatment methods/goals should not necessarily be taken as limiting.

### Kits and Articles of Manufacture

In some embodiments, the invention provides a kit or an article of manufacture containing materials useful for the methods described herein and/or comprising examples of the systems/devices described herein. Such kits can optionally comprise one or more containers, labels, and instructions, as well components for monitoring of treatment.

The kits can also optionally comprise one or more light sources, polarizers, lenses, polarization rotators, fiber optics, light detectors, computers, etc. as well as optionally other components. The kits can optionally include scaffolding, armature or other organizational structures to controllably position and/or move the various components of the systems/devices of the invention.

In many embodiments, the kits comprise instructions (e.g., typically written instructions) relating to the use of the kit to determine and/or monitor changes in tissue (e.g., collagen). In some embodiments, the kits comprise a URL address or phone number or the like for users to contact for instructions or further instructions.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Measurement of DoLP and θ

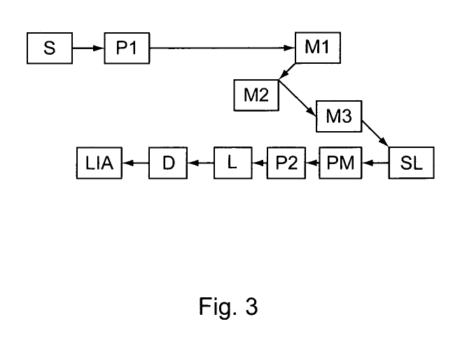

The instrument configuration diagrammed in FIG. 3 was used to perform measurements on several sample types (i.e., mirror, half wave plate, and bovine tendons). In FIG. 3 the system components comprise: detector D (a silicon photodiode with built-in trans-impedance amplifier), lens L (having a 1″ diameter and 75 mm FL), lock-in amplifier LIA (With reference to source modulation. As described below, the source is modulated at 1 kHz. This modulation signal is also supplied as a reference signal for the lock-in amplifier.), mirror M1 (0.5″ diameter round), mirror M2 (0.5″ diameter round), mirror M3 (1.0″ diameter D-shaped), polarizer P1 (source polarizer), polarizer P2 (source polarizer), parabolic mirror PM (90 degree off-axis), laser source S (808 nm, 200 mW, 1 kHz, square-wave modulated), and sampling lens SL (a split lens, 1.0″ diameter PCX, 15 mm FL, 1 mm black ABS spacer).

In the measurements, the input polarization angle was maintained at vertical (0 degrees). The orientation angle of the input polarization relative to the sample optic axis was changed by manually rotating the sample on top of the sampling lens (SL). The Stokes vector values were measured by manually rotating the polarizer (P2) in front of the detector, and manually recording the lock-in amplifier readings.

The “mirror” sample was a 1″ mirror suspended about 5 mm above the sampling lens. The “HWP” sample was a half wave plate placed directly over the sampling lens with a ½″ mirror placed on top of the half wave plate. The “Teflon” sample was an 8 mm thick, 1.5″ diameter disk of Teflon placed directly on top of the sampling lens. The “bovine tendon” sample was suspended about 3 mm above the sampling lens.

TABLE 1

| Sample | DoLP (0°) | DoLP (45°) | θ(0°) | θ(45°) |
|---|---|---|---|---|
| Mirror | 0.925 | 0.937 | 4.3° | 4.2° |
| HWP | 0.956 | 0.581 | 3.6° | −9.2° |
| Teflon | 0.151 | 0.168 | 5.8° | 5.2° |
| Bovine | 0.121 | 0.060 | 1.3 | −5.5 |
| tendon | | | | |
| Bovine | 0.111 | 0.064 | 3.9 | 3.1 |
| tendon | | | | |
| Bovine | 0.112 | 0.052 | 2.6 | −4.8 |
| tendon | | | | |

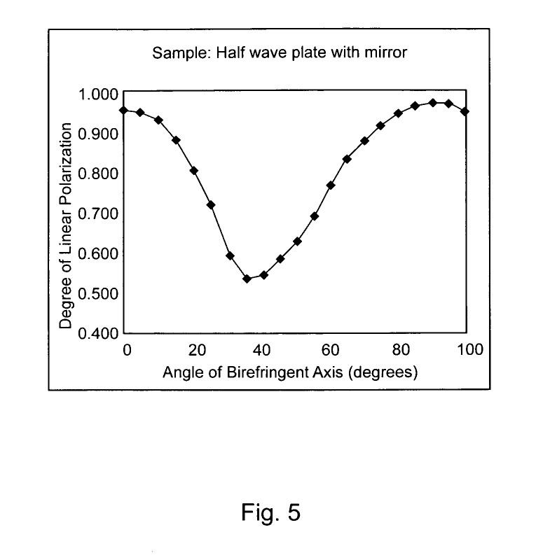

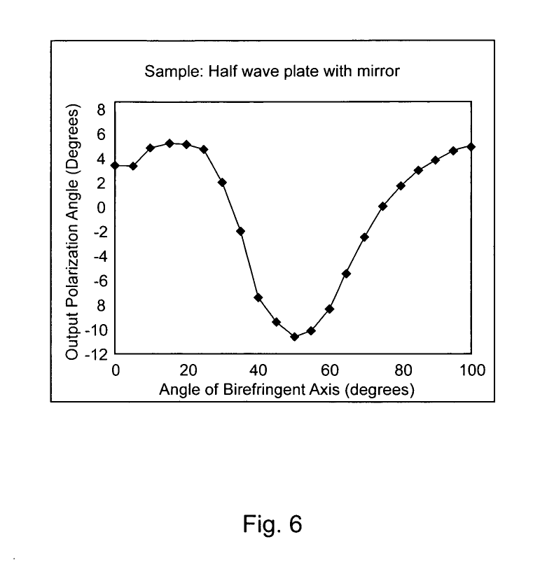

The results are summarized in Table 1. As expected, the “mirror” sample was unaffected by rotation relative to the input polarization. In contrast the “HWP” sample was strongly orientation dependent: both DoLP and θ decreased when the optic axis was rotated from 0 to 45 degrees. For this sample, the response was also measured at orientations between 0 and 100 degrees, in 5 degree increments. The results are displayed in FIGS. 4-6. As can be seen in FIGS. 5 and 6, both DoLP and θ could be used to readily identify the optic axis of this sample. The slight difference in the location of the optic axis as determined by the two methods is thought to be due to the known depolarization artifact seen previously at polarization angles other than multiples of 90 degrees. See, e.g., “Orientation of Monitoring Polarization Relative to Collagen Axis” above. The fact that the θ (45° polarization rotation is less than the expected 90 degrees is also indicative that a polarization artifact needs correcting. The instrument arrangement depicted in FIG. 2 is thought to be able to compensate for this artifact.

Also in this example, Teflon was used to test the effect of a non-birefringent but strongly scattering material. The DoLP in Table 1 indicates that the scattering in Teflon resulted in substantial depolarization of the laser light, but no orientation dependence was observed either for DoLP or θ.

The three bovine tendon measurements shown in Table 1 were performed on the same piece of tendon, but with re-hydration and repositioning of the sample between measurements. Although the detected light was substantially depolarized at both orientations, a reproducible reduction in DoLP from the 0 degree orientation to the 45 degree orientation was observed. In 2 of the 3 measurements, a substantial change in polarization rotation (θ) was also observed. It should be noted that not all bovine tendon samples that were tried showed significant orientation effects.

### Example 2

### Automated DoLP Measurement

The instrument outlined in FIG. 7 was constructed to automate collagen birefringence measurement. The arrows in the Figure indicate the direction of the optical beam. The system components in FIG. 7 include: detector D (silicon photodiode with built-in trans-impedance amplifier), lens L (1″ diameter, 75 mm FL), lock-in amplifier LIA (reference to source modulation), mirror M1 (0.5″ diameter round), mirror M2 (0.5″ diameter round), mirror M3 (1″ diameter, D-shaped), polarizer P1 (source polarizer), polarizer P2 (source polarizer), parabolic mirror PM (90 degree off-axis), polarization rotator PR (liquid crystal), rotation stage R (for orienting sample), laser source S (808 nm, 200 mW, 1 kHz, square-wave modulated), and sampling lens SL (split lens, 1″ diameter PCX, 15 mm FL, 1 mm black ABS spacer). As can be seen, the configuration in this Example is similar to that in Example 1, but with the addition of the rotation stage and the polarization rotator.

A LabView (National Instruments, Austin, Tex.) computer program was written to sequentially set the voltage of the liquid crystal polarization rotator (PR in FIGS. 7) to 4 levels, corresponding to linear polarization rotations of 0, 45, 90, and 135 degrees. At each PR voltage the signal detected on the lock-in amplifier LIA was recorded on a computer. After completion of the measurements at the 4 polarization settings, the Degree of Linear Polarization (DoLP) was automatically calculated (see equations 2-5 above). The rotation stage R was then manually rotated by 10 degree increments over a 90 degree range, and the above-described procedure was repeated. This process was continued until a plot of DoLP vs. sample orientation angle was complete. A flowchart outlining the software is shown in FIG. 14.

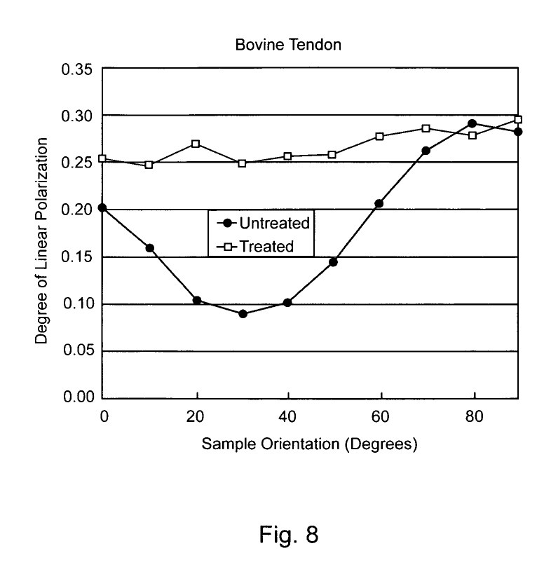

An example measurement is shown in FIG. 8. It will be appreciated that the measurement of the sample orientation in FIG. 8 could also be a measurement of PR1 orientation. The sample used in the measurement was bovine tendon, stored frozen and then thawed to room temperature just prior to measurement in a bath of phosphate buffered saline (PBS). The tendon sample was held in a sample well that was filled with PBS during the measurement. The bottom of the sample well consisted of a 2 mm thick fused silica window held just above sampling lens SL. The window was held within rotation stage R that was manually rotated to adjust the sample orientation. As can be seen in FIG. 8, the birefringent axis of the untreated sample was readily discerned by plotting the DoLP vs. the sample orientation angle. As discussed above in the specification, it is expected that the minimum DoLP will be observed when the input linear polarization is at 45 degrees relative to the birefringent axis of the collagen. A clear minimum occurred at a sample orientation of approximately 30 degrees in FIG. 8. The maximum in the DoLP is theoretically expected to occur 45 degrees from the minimum (i.e., where input polarization is parallel to light birefringence of the collagen). The experimentally observed maximum at a sample orientation of 80-90 degrees was in agreement with this expectation. The minima (and maxima) as a function of sample orientation were expected to recur at multiples of 90 degrees, and this was also verified in other experiments (data not shown).

Collagen birefringence is known to diminish with heating/denaturation. The “untreated” bovine tendon sample shown in FIG. 8 was therefore “treated” by heating it in a microwave oven while immersed in PBS. Its birefringence was then re-measured. The resultant DoLP curve (squares in FIG. 8) was nearly flat; suggesting that the “treated” sample was almost fully denatured. Taken together, these observations show that the birefringent axis of collagen can be determined with high accuracy by embodiments of the invention. It should be noted from the Figure that the maximum DoLP was relatively unchanged by treatment. This observation supports the idea (discussed above) that by monitoring the DoLP at its maximum value, the measurement of an underlying planar collagen structure could be achieved; whereas by monitoring the DoLP at its minimum value, maximum sensitivity to treatment of linearly oriented collagen structures is achieved.

### Example 3

### Tendon Birefringence Measurements Through Phantom Skin Layers

Example 3 demonstrates measurement of tendon birefringence through an intervening layer. The ability of measuring birefringence through an intervening layer (e.g., skin) is important in, e.g., non-invasive orthopaedic applications. In this example several skin “phantoms” were tested. These phantom materials included gelatin, Teflon® (DuPont, Wilmington, Del.) and Intralipid® (Kabivitrum, Alameda, Calif.) mixed with gelatin. Gelatin was used because it is composed of denatured collagen, the primary protein constituent found in skin. Teflon was used due to its ready availability in multiple thicknesses and the fact that, like skin, it is highly scattering and weakly absorptive at 808 nm. However, unlike skin, Teflon has little or no natural birefringence. 2% Intralipid in gelatin was used as a phantom mimicking the primary constituents of tissue (water, collagen, and fat) and having similar scattering properties to skin. See, e.g., T. L. Troy and S. N. Thennadil, “Optical Properties of Human Skin in the NIR Wavelength Range of 1000 to 2200 nm,” J. Biomed. Opt., 6:167 (2001). The measurements performed in Example 3 were done on the same system arrangement as that of Example 2.

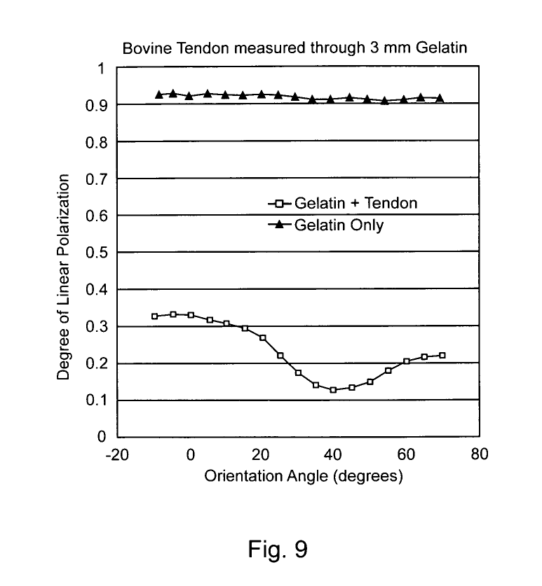

FIG. 9 shows the results of birefringence measurement for a 3 mm thick layer of gelatin both without (diamonds) and with (squares) a bovine tendon placed on top of the gelatin. The gelatin sample was first placed in the sampling well for the former measurement; a section of bovine tendon was then placed on top of the Teflon for the later measurement. In the absence of the tendon, the degree of linear polarization was seen to be high (>0.9) at all orientation angles, and showed only weak orientation dependence. With the tendon on top of the gelatin, the degree of linear polarization was seen to be lower at all orientation angles, and showed an angular dependence similar to that of the tendon alone (see FIG. 8 above). Additional experiments with greater thicknesses of gelatin (data not shown) demonstrated that the tendon birefringence could still be readily measured through thicknesses of at least 7 mm. These experiments showed that the presence of denatured collagen in a low-scattering matrix posed little challenge for the tendon measurement; both the amplitude of the DoLP change and the signal to noise ratio were similar whether the gelatin layer was present or absent.

Teflon was also examined as an intervening “skin” phantom layer. A series of different thicknesses of Teflon, ranging in thickness from 0.1 to 5 mm, were used. For each experiment, the Teflon sample was first placed in the sampling well and measured alone to verify that no significant birefringence effect was observed. After such measurement, a section of bovine tendon was placed on top of the Teflon, weighed down to keep the layers in close contact with each other, and a second measurement was performed. In addition to varying the thickness of Teflon through which the measurement was made, the gap between the sampling lens and the sampling well was varied in different experiments. Increasing this gap had the effect of increasing the overlap between the source beam and the detection cone, which in turn affected the depth of penetration of the measurement.

A measurement of tendon birefringence through a 0.5 mm thickness of Teflon is shown in FIG. 10. The air gap between the sampling lens and sample window was set at 3 mm for this measurement. Also, the Degree of Linear Polarization was split into 2 parts: the Degree of Vertical Polarization (DoVP) and the Degree of Horizontal Polarization (DoHP). DoVP and DoHP were defined as:

$$\begin{matrix}DoVP = \frac{Q}{I} & Equation 7 \\ DoHP = \frac{U}{I} & Equation 8\end{matrix}$$

where Q, U, and I are as defined above in the specification. Determination of the amplitude of the change in the degree of polarization with sample orientation was found to be more reliable by simultaneously fitting the DoVP and DoHP curves than by fitting the DoLP curve alone. The DoVP and DoHP curves were fit to sine functions, with the angle set to 4 times the orientation angle, and further constrained to have relative phases separated by 45 degrees and the same amplitude.

Comparing FIGS. 8 and 10, it can be seen that the change in the degree of polarization is much smaller when Teflon separates the tendon from the sampling optics. This is due to the strong light scattering within the Teflon which depolarizes the light before it reaches the tendon and again after it is reflected back from the tendon. The presence of a strongly scattering layer (such as Teflon) constitutes a much greater measurement challenge than a weakly scattering layer containing denatured collagen (such as gelatin).

A measurement of tendon birefringence was also made through a skin phantom that had been reported to have similar scattering properties to human skin; namely 2% Intralipid. The Intralipid was embedded in gelatin in order to make a solid layer upon which a bovine tendon sample could be placed. Shown in FIG. 11 is the difference in the degree of polarization, through a 2.2 mm thick layer of Intralipid, between the presence or absence of an overlying layer of bovine tendon. The DoVP and DoHP curves were simultaneously fit (solid lines in FIG. 5) as described above, and a comparison of the fit amplitude with the fitting error indicated that there was a significant trend in the angular variation in the DoP due to the bovine tendon.

### Example 4

### Dermal Collagen Birefringence Measurements Through Epidermis Layers

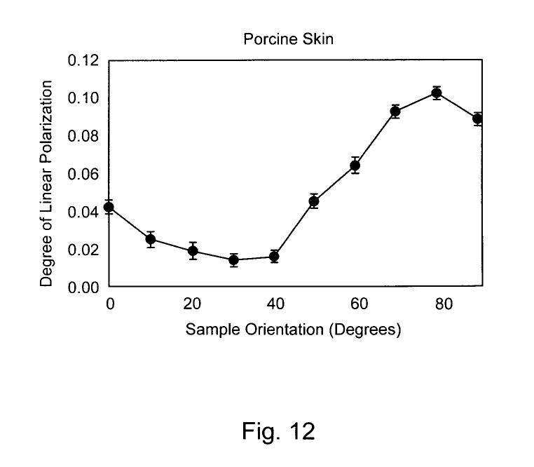

A measurement of birefringence of dermal collagen through an actual skin layer was done for Example 4. The component arrangement in Example 4 was the same as that shown in FIG. 7 and the example was carried out in the same manner as the tendon measurement shown in FIG. 8. The sample measured was from an adult pig and contained a full thickness of skin including epidermis, dermis, and subcutaneous fat. The epidermis side was placed on the sampling window and the birefringence was measured through the epidermis. The results of such measurement are shown in FIG. 12. This measurement demonstrates that embodiments of the invention were able to measure the dermal collagen birefringence through the epidermis. The error bars shown on the plot in FIG. 12 were computed from 5 measurements on the same sample. Even though the epidermal layer on an adult pig is typically thicker than human skin, pig skin is used in many experiments and in many fields as a model for human skin due to its similarity.

### Example 5

### Repeated Porcine Dermal Collagen Birefringence Measurements During Thermal Treatment

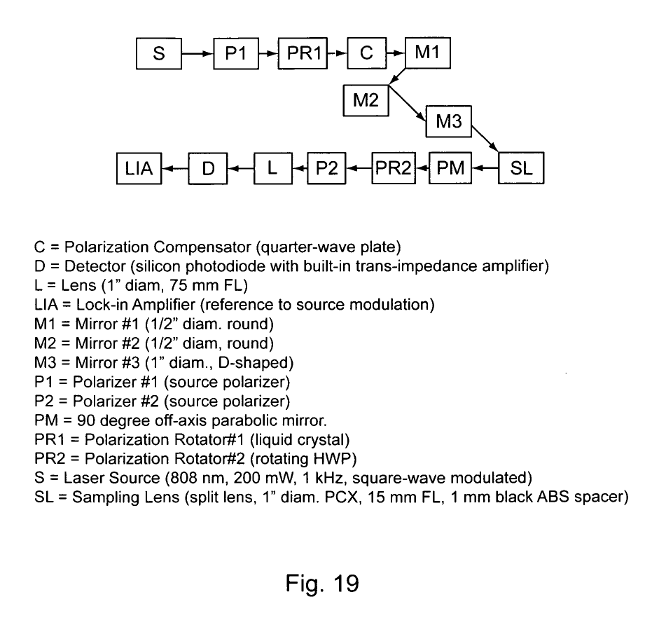

For example 5, the birefringence of porcine skin was repeatedly measured as it was undergoing thermal treatment. The instrument was configured as diagrammed in FIG. 19, allowing the measurements to be made under automation. Compared to the above-described configuration for automated measurement (FIG. 13), the locations of the liquid crystal (LC) and rotating half-wave plate (HWP) rotators have been swapped, and the quarter-wave compensator plate (C) has been moved from the detection to the excitation side of the optical train. The reason for the change is that the LC device was found to function purely as a linear polarization rotator only when the incoming light had a linear polarization angle of 45 degrees relative to the LC birefringent axis. At angles other than 45 degrees the LC device also converted linear polarized light into circularly polarized light. This is in contrast to the HWP which acts purely as a linear polarization rotator at all input linear polarization angles. The angle of rotation is twice the angle between the incoming polarization and the birefringent axis of the HWP. By placing the LC device on the excitation side of the optical train, the angle of the light entering the LC could be fixed at 45 degrees for all measurements.

Fresh porcine skin was purchased from a local butcher and used on the same day (without freezing). Samples included some subcutaneous fat. A roughly 20 mm disk of skin was cut out with scissors and held in place with elastic straps with the skin side facing the window. The window was glued onto a 1″ OD plastic tube to make a small (˜10 mL) sample chamber. The sample chamber was filled with phosphate buffered saline (PBS) and a resistive heater and thermocouple attached to an aluminum disk were immersed in the PBS, above the sample. The resistive heater and thermocouple were attached to a temperature controller. A second thermocouple was placed inside the fat layer of the skin sample to monitor the actual skin temperature. A small stirring device was used to help maintain a uniform temperature within the heating bath.

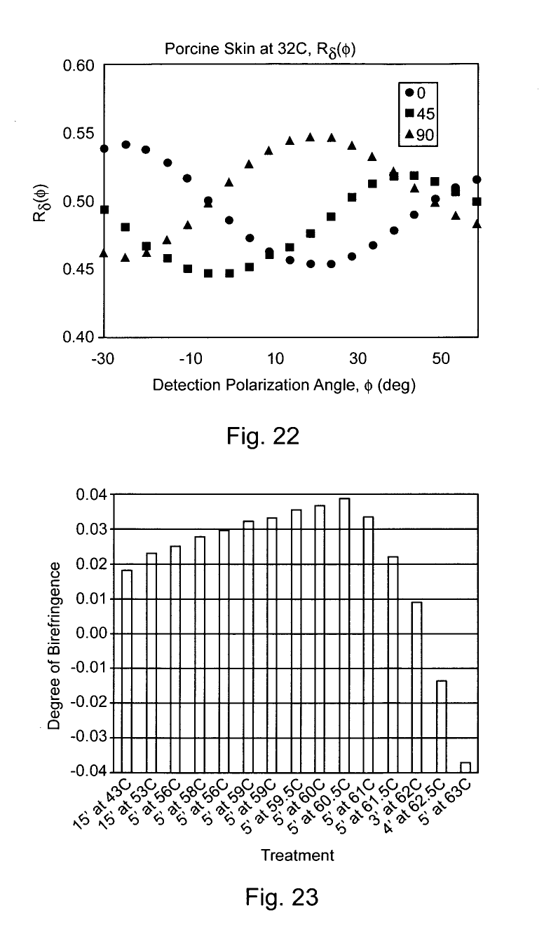

The DoP function used for this example is shown in Equation 22. This function is a ratio of I_{δ} (φ)/1 measurements collected with the sample orientation, γ, set at 0 (FIG. 20) and 45 degrees (FIG. 21). I₀/I, I₄₅/I, and I₉₀/I are plotted as a function of the detection polarization angle, φ, in FIGS. 20 and 21, while the result of computing the ratio, R_{δ} (φ), is shown in FIG. 22. With the angular dependence thus established, one δ angle (δ=0) and two Dangles (δ=−25° and 20°) were then selected at which to monitor birefringence. As can be seen in FIG. 22, for the selected δ angle (blue circles), the selected φ angles correspond to the maximum and minimum values of R_{δ}(φ). The degree of birefringence (DoB) was thus evaluated according to:

DoB=R_{δ=0}(φ=−25°)−R_{δ=0}(φ=20°) Equation 23

The temperature of the porcine skin sample was raised in steps and monitored. The resulting DoB following each treatment step is shown in FIG. 23. Raising the skin temperature from 43 C up to about 60 C resulted in a gradual increase in the birefringence followed by an abrupt decrease in the birefringence between 60 and 63 C. The transition temperature at which the birefringence began to decrease (60 C) is in good agreement with prior measurements of the transition temperature for collagen contraction as a function of temperature (for example, see FIG. 2 in M. S. Wall et al., “Thermal Modification of Collagen”, J. Shoulder Elbow Surg., July/August 1999, p. 339-344).

### Example 6

### Human In Vivo Dermal Collagen Birefringence Measurements

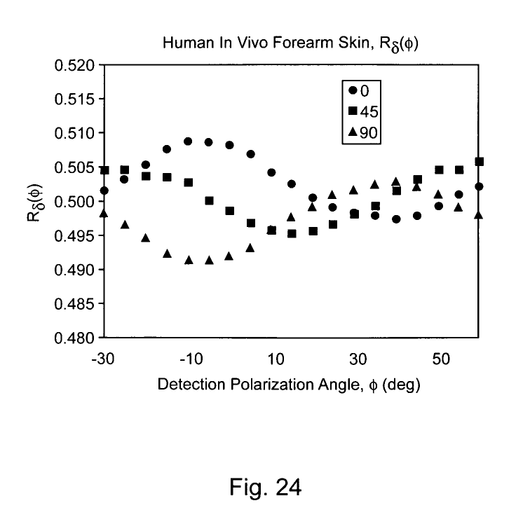

A non-invasive measurement of the birefringence of skin on the forearm of a live subject is shown in FIG. 24. The instrument configuration and algorithms for computing the DoP are the same as in Example 5. The dependence of the DoP function, R_{δ}(φ), on the detection polarization angle is readily apparent, indicating that changes in collagen birefringence could be assessed with high sensitivity.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1.-56. (canceled)

57. A method of monitoring a change in birefringence status of a biological structure, the method comprising:
a) determining a first angular dependence of a birefringence of the biological structure relative to an input polarization by exposing the biological structure to a first light at a first polarization angle; measuring a reflected birefringence that is reflected from the structure at the first polarization angle at L₄₅, L₊₄₅, I₀, and I₊₉₀, wherein I_{φ} is the reflected birefringence measured by a detection polarizer at polarization angle φ; computing Q, U, and I from the measured reflected birefregence, wherein Q=I₀−I₉₀, U=I₊₄₅−I₋₄₅, and I=I₀+I₉₀; computing a DoLP, θ, DoVP, or DoHP from the measured reflected birefringence, wherein
$$DoLP = \frac{\sqrt{{Q}^{2} + {U}^{2}}}{I} ;$$
2θ=tan⁻¹(U/Q);
$$DoVP = \frac{Q}{I} and DoHP = \frac{U}{I} ;$$
exposing the biological structure to a second light at a second polarization angle, which second angle is greater than the first angle; and, repeating the exposing over a range of at least 5 degrees;
b.) applying a treatment to the biological structure;
c.) determining a second angular dependence of a birefringence of the biological structure relative to an input polarization by exposing the biological structure to a first light at a first polarization angle; measuring a reflected birefringence that is reflected from the structure at the first polarization angle at L₄₅, L₊₄₅, I₀, and I₊₉₀, wherein I_{φ} is the reflected birefringence measured by a detection polarizer at polarization angle φ; computing Q, U, and I from the measured reflected birefregence, wherein Q=I₀−I₉₀, U=I₊₄₅−I₋₄₅, and I=I₀+I₉₀; computing a DoLP, θ, DoVP, or DoHP from the measured reflected birefringence, wherein
$$DoLP = \frac{\sqrt{{Q}^{2} + {U}^{2}}}{I} ;$$
2θ=tan⁻¹(U/Q);
$$DoVP = \frac{Q}{I} and DoHP = \frac{U}{I} ;$$
exposing the biological structure to a second light at a second polarization angle, which second angle is greater than the first angle; and, repeating the exposing over a range of at least 5 degrees;
d.) comparing the first angular dependence and the second angular dependence wherein a difference between the first angular dependence and the second angular dependence indicates a change in birefringence status of the biological structure.

58. The method of claim 57, wherein a change in birefringence status corresponds to a structural change in the biological structure.

59. A method of determining the degree of birefringence of a biological structure, the method comprising:
a) exposing the structure to light at a first input polarization state, with the orientation of the structure at a first sample orientation state;
b) measuring a first light intensity that is reflected from the structure at a first output polarization state;
c) varying at least one of the input polarization state, output polarization state, or sample orientation state, to at least one additional state and measuring the light intensity at each additional state;
d) computing the degree of polarization by combining at least two light intensity measurements at two different states;
e) relating the degree of polarization to the degree of birefringence;
wherein the degree of birefringence of the biological structure is determined.

60. The method of claim 59, wherein the degree of birefringence is used to monitor a treatment of biological structure.

61. The method of claim 60 wherein treatment comprises one or more of:
application of physical energy, application of radio frequency waves, application of ultrasound, application of heat, application of cold, or application of a cosmeceutical.

62. The method of claim 59, wherein the biological structure comprises a collagen structure.

63. The method of claim 62, wherein the biological structure comprises skin, a fascia, an aponeurosis, a tendon, a ligament, a capsule, a fascia, a vascular wall, a vaginal wall, an introitus, an aponeourosis, or a urethra.

64. The method of claim 59, wherein the light exposed to the biological structure traverses through a second biological structure before it exposes the biological structure.

65. The method of claim 59, wherein measurements of the degree of birefringence at multiple said states are used to distinguish between a collagen layer with at least partially planar structure from a collagen layer with at least partially unidirectional strand structure.

66. The method of claim 59, wherein the first input polarization state is linear polarized light with a particular orientation.

67. The method of claim 59, wherein the first input polarization state is circularly polarized light.

68. A system or device for monitoring the degree of birefringence in one or more biological structures in a tissue, the system or device comprising:
a light source component configured to emit light to the tissue;
one or more light polarizer components configured to alter the polarization state of the light emitted from the light source or reflected from the tissue;
a light detection component configured to detect light reflected from the tissue; and,
a computer or processor component, wherein the computer or processor component comprises an instruction set programmed to:
direct the detection component to measure a first reflected light at a particular polarization state from the one or more collagen structures following exposure of the light source to the tissue to light at a particular polarization state;
direct the detection component to measure at least one additional reflected light at a particular polarization state from the one or more collagen structures following exposure of the light source to the tissue to light at a particular polarization state;
use the first reflected light and at least one additional reflected light to compute the degree of birefringence, thereby monitoring the degree of birefringence in one or more tissue structures; and,
output the degree of birefringence monitored to a user.

69.-70. (canceled)
